(19) United States
(12) Patent Application Publication
(10) Pub. No.: US 2003/0180352 A1
(43) Pub. Date: Sep. 25, 2003

(54) Solid carriers for improved delivery of active ingredients in pharmaceutical compositions

(76) Inventors: Mahesh V. Patel, Salt Lake City, UT (US); Feng-Jing Chen, Salt Lake City, UT (US)

Correspondence Address: REED & ASSOCIATES, 800 MENLO AVENUE, SUITE 210, MENLO PARK, CA, 94025, US

(21) Appl. No.: 10/159,601

(22) Filed: May 30, 2002

Related U.S. Application Data
(63) Continuation-in-part of application No. 09/800,593, filed on Mar. 6, 2001.
Application No. 09/800,593 is a division of application No. 09/447,690, filed on Nov. 23, 1999, now Pat. No. 6,248,363.

(51) Int. Cl. A61K 31/4439; A61K 9/20

(52) U.S. Cl. 424/465; 514/338

(57) ABSTRACT

The present invention provides solid pharmaceutical compositions for improved delivery of a wide variety of active ingredients contained therein or separately administered. In one embodiment, the solid pharmaceutical composition includes a solid carrier, the solid carrier including a substrate and an encapsulation coat on the substrate. The encapsulation coat can include different combinations of active ingredients, hydrophilic surfactant, lipophilic surfactants and triglycerides, and solubilizers. In another embodiment, the solid pharmaceutical composition includes a solid carrier, the solid carrier being formed of different combinations of active ingredients, hydrophilic surfactants, lipophilic surfactants and triglycerides, and solubilizers. The compositions of the present invention can be used for improved delivery of active ingredients.

CROSS REFERENCE To RELATED APPLICATIONS

This application is a continuation-in-part of U.S. Ser. No. 09/800,593 filed on Mar. 6, 2001, which is a divisional of U.S. Ser. No. 09/447,690, filed on Nov. 23, 1999, now issued as U.S. Pat. No. 6,248,363.

### FIELD OF THE INVENTION

The present invention relates to pharmaceutical delivery systems for pharmaceutical active ingredients, such as drugs, nutritionals, cosmeceuticals, and diagnostic agents. In particular, the present invention provides compositions and dosage forms including solid carriers for improved delivery of pharmaceutical active ingredients.

### BACKGROUND OF THE INVENTION

Hydrophobic active ingredients, such as progesterone, cyclosporin, itraconazole and glyburide present delivery challenges due to their poor aqueous solubility and slow dissolution rate. Several commercial products of these hydrophobic drugs are available, the various products using different methods to try to enhance in vivo performance. One approach is size reduction by micronization, such as in Prometrium (micronized progesterone) and Micronase (micronized glyburide). Other approaches include size reduction in emulsion formulations, such as in Sandimmune (cyclosporin emulsion) and NeOral (cyclosporin microemulsion). These approaches suffer from several disadvantages. Micronization/nanonization presents processing and stability challenges, as well as dissolution limitations, since the micronized/nanosized drug still possesses a high degree of crystallinity. Liquid formulations present drug precipitation and packaging challenges, due to solvent evaporation. Moreover, non-solid formulations are more prone to chemical instability and capsule-shell incompatibility, leading to the possibility of leakage upon storage.

Solid carriers for pharmaceutical active ingredients offer potential advantages over micronized drugs, emulsions or solubilized formulations. Solid carriers, typically of size less than about 2 mm, can easily pass through the stomach, thus making the performance less prone to gastric emptying variability. Further, the problems of leakage and other disadvantages of liquid formulations are not present in solid carrier formulations. To date, however, such solid carrier formulations generally have been limited to a few specific drugs, due to difficulties in formulating appropriate drug/excipient compositions to effectively coat the active ingredient onto a carrier particle.

Conventional solid dosage forms of hydrophobic active ingredients, such as tablets, or multiparticulates in capsules, often exhibit slow and incomplete dissolution and subsequent absorption. These formulations often show a high propensity for biovariability and food interactions of the active ingredient, resulting in restrictive compliance/labeling requirements.

Due to the slow dissolution and dependence on gastric emptying, solid dosage forms often delay the onset of some hydrophobic active ingredients.

In addition, it is well known that active ingredients that are acid-labile, will degrade quickly when placed in contact with acids. An enteric coating therefore is typically applied to a core containing such acid-labile drugs was applied to prevent the drug from contacting the acidic pH conditions of the stomach upon oral administration. The acidic residue of the enteric coating, however, can degrade the acid-labile drug during storage. To solve this problem, a significant amount of inorganic alkaline materials were introduced to the core by specific processes, such as granulation so as to ensure the acid-labile drug being evenly in contact with the basic inorganic salt.

Thus, there is a need for pharmaceutical compositions and dosage forms, and methods therefor, that do not suffer from the foregoing disadvantages. Further, there remains a need for stable formulations of active ingredients such as acid-labile compounds, that have minimal limitations with regards to processing techniques or material selection.

### SUMMARY OF THE INVENTION

It is an object of the invention to provide solid pharmaceutical compositions having active ingredients in a rapid dissolvable and more solubilized state therein.

It is another object of the invention to provide solid pharmaceutical compositions having more rapid dissolution upon administration to a patient.

It is another object of the invention to provide solid pharmaceutical compositions having more sustained and complete solubilization upon administration to a patient.

It is another object of the invention to provide solid pharmaceutical compositions capable of delivery a wide variety of pharmaceutical active ingredients.

It is another object of the invention to provide solid pharmaceutical compositions of coated substrate materials without the need for binders.

It is another object of the invention to provide solid pharmaceutical compositions having increased chemical stability of the active ingredient

It is another object of the invention to provide solid pharmaceutical compositions having increased chemical stability of the active ingredient upon prolonged storage.

It is another object of the invention to provide solid pharmaceutical compositions having increased chemical stability of the active ingredient in the gastrointestinal tract upon administration.

It is another object of the invention to provide solid pharmaceutical compositions remaining stable upon exposure to moisture and/or acidic species, such as protons.

It is another object of the invention to provide solid pharmaceutical compositions having increased chemical stability of the active ingredient, without the need for the active ingredient to be evenly in contact with a basic inorganic salt.

It is another object of the invention to provide solid pharmaceutical compositions having stable active ingredients, without the need for the inclusion of a basic inorganic salt in the composition.

It is another object of the invention to provide solid pharmaceutical compositions having stable active ingredients, without the need for an enteric coating.

It is another object of the invention to provide solid pharmaceutical compositions having minimal limitations with regard to processing techniques or material selection.

It is another object of the invention to provide solid pharmaceutical compositions having no need to be processed by granulation, particularly wet granulation.

It is another object of the invention to provide solid pharmaceutical compositions that do not require water or organic solvents during manufacture, which might be detrimental to the stability of the active ingredient.

It is another object of the invention to provide solid pharmaceutical compositions having a substantially hydrophilic solid carrier and containing at least one excipient selected from the group consisting of hydrophilic surfactants and solubilizers.

It is another object of the invention to provide solid pharmaceutical compositions having a solid carrier that is substantially free of “hard fat” or “adepo solidus”, which is a mixture of triglycerides, diglycerides and monoglycerides, and has a melting point of 30-45° C. (e.g. Witepsol H15) (European Pharmacopoeia 1997).

It is another object of the invention to provide solid pharmaceutical compositions capable of improving the absorption and/or bioavailability of pharmaceutical active ingredients.

It is another object of the invention to provide solid pharmaceutical compositions having better protection of the upper gastrointestinal tract from untoward effects of the active ingredient.

It is another object of the present invention to provide solid pharmaceutical compositions capable of improving the palatability of or masking the taste of unpalatable pharmaceutical active ingredients.

In accordance with these and other objects, the present invention provides solid pharmaceutical compositions for improved delivery of a wide variety of pharmaceutical active ingredients contained therein or separately administered.

In one embodiment, the solid pharmaceutical composition includes a solid carrier, the solid carrier including a substrate and an encapsulation coat on the substrate. The encapsulation coat includes at least one ionic or non-ionic hydrophilic surfactant. Optionally, the encapsulation coat can include an active ingredient, a lipophilic component such as a lipophilic surfactant or a triglyceride, or both an active ingredient and a lipophilic component.

In another embodiment, the solid pharmaceutical composition includes a solid carrier, the solid carrier including a substrate and an encapsulation coat on the substrate. The encapsulation coat includes a lipophilic component, such as a lipophilic surfactant or a triglyceride. Optionally, the encapsulation coat can include an active ingredient, an ionic or non-ionic hydrophilic surfactant, or both an active ingredient and a hydrophilic surfactant.

In another embodiment, the solid pharmaceutical composition includes a solid carrier, the solid carrier including a substrate and an encapsulation coat on the substrate. The encapsulation coat includes an active ingredient and an ionic or non-ionic hydrophilic surfactant; an active ingredient and a lipophilic component such as a lipophilic surfactant or a triglyceride; or an active ingredient and both a hydrophilic surfactant and a lipophilic component.

In another embodiment, the solid pharmaceutical composition includes a solid carrier, wherein the solid carrier is formed of at least two components selected from the group consisting of active ingredients; ionic or non-ionic hydrophilic surfactants; and lipophilic components such as lipophilic surfactants and triglycerides.

In yet another embodiment, the pharmaceutical composition in the form of a solid carrier comprising an admixture of a therapeutically effective amount of an active ingredient; and at least one excipient selected from the group consisting of: a hydrophilic surfactant; at least one lipophilic additive selected from the group consisting of lipophilic surfactants, triglycerides, and combinations thereof; and a solubilizer.

In other aspects, the present invention also provides dosage forms of any of the solid pharmaceutical compositions, and methods of using the solid pharmaceutical compositions.

These and other objects and features of the present invention will become more fully apparent from the following description and appended claims, or may be learned by the practice of the invention as set forth hereinafter.

### BRIEF DESCRIPTION OF THE DRAWING

In order to illustrate the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 is a graph showing the extent of dissolution/release of glyburide as a function of time for a composition according to the present invention and two prior art compositions.

FIG. 2A is a graph showing the extent of dissolution/release of progesterone as a function of time for two compositions according to the present invention and the pure bulk drug.

FIG. 2B is a graph showing the extent of dissolution/release of progesterone as a function of time for two compositions of the present invention, a conventional commercial formulation of progesterone, and the pure bulk drug.

FIG. 3 is a graph showing the extent of dissolution/release of omeprazole as a function of time for two compositions according to the present invention and a prior art composition.

### DETAILED DESCRIPTION OF THE INVENTION

It is to be understood that unless otherwise indicated, this invention is not limited to specific active agents, vehicles, excipients, dosage forms, or the like, as such may vary. It is also to be understood that the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “an active agent” includes a single active agent as well a two or more different active agents in combination, reference to “an excipient” includes mixtures of two or more excipients as well as a single excipient, and the like.

The present invention provides solid pharmaceutical compositions for improved delivery of a wide variety of pharmaceutical active ingredients, contained therein or separately administered in variety of aspects. The solid carrier of the present invention can be utilized for improving the performance, safety, efficacy and/or patient compliance of the active ingredient or combinations of active ingredients, such as: sustained therapeutic effect; targeted gastrointestinal site delivery for local or systemic effects to the stomach, duodenum, ileum, jejunum, colon or rectum; reduced lingering side effects; increased bioavailability; improved solubility, membrane permeability, and/or in vivo/ex vivo stability of the active ingredient; reduced variability in absorption of the active ingredient; reduced food effect; protection of the gastrointestinal tract; reduced irritating effect caused by the active ingredient; taste masking or odor masking; flavor augmentation; and improved physical and/or chemical stability of the active ingredient and/or the dosage form during storage.

The accomplishment of one or more of the above-mentioned improvements can be satisfied by providing the solid carrier of the present invention with a variety of release profiles for the active ingredient or combination of active ingredients, including: immediate release, delayed release, sustained or extended release, pulsatile release, multiple stage release, targeted release, chronometric, timed release and combinations thereof. For example, for an acid-labile proton pump inhibitor (e.g. lansoprazole), a delayed release dosage form will protect the active ingredient from the acidic gastric fluids in the stomach, and will also offer protection during storage. For another example, for a sleep aid (e.g. zolpidem) having an intrinsically slow onset and lingering effect, an immediate release dosage form will provide the active ingredient with the desirable fast-in and fast-out characteristics of systemic delivery to both optimize the therapeutic effect as well as reduce the lingering side effect. For another sleep aid (e.g. zaleplon) having an intrinsically fast elimination, a sustained release or pulsatile release to mimic two or more bolus doses will provide the advantages of preventing the therapeutic effect of the active ingredient to wear off between doses, and of improving patient compliance. Similarly, the same principle can be applied to a migraine drug (e.g. sumatriptan) and a gastrointestinal agent (e.g. ondansetron) with the similar desirable PK profiles. A suitable taste masking application could be an antibiotic, and a suitable example of targeted site release would be budesonide.

In one embodiment, the solid pharmaceutical composition includes a solid carrier, the solid carrier including a substrate and an encapsulation coat on the substrate. The encapsulation coat can include different combinations of active ingredients, hydrophilic surfactants, lipophilic additives (lipophilic surfactants and triglycerides), and solubilizer. In another embodiment, the solid pharmaceutical composition includes a solid carrier, the solid carrier being formed of the active ingredient and different combinations of hydrophilic surfactants, lipophilic additives and solubilizers. In general, the hydrophilic surfactant and lipophilic additive, or the lipophilic additive alone, provide a moisture barrier, while the solubilizer functions as a disintegration or dissolution aid to provide a desirable release profile of the active ingredient from the formulation once it reaches the small intestine. These examples are merely illustrative, and it must be emphasized that any given drug identified by structural or functional class may be replaced with another drug of the same structural or functional class.

In another embodiment, the solid pharmaceutical composition includes a solid carrier, the solid carrier including a substrate and an encapsulation coat on the substrate. The encapsulation coat can include different combinations of acid-labile active ingredients, hydrophilic surfactants, lipophilic additives, and solubilizer. In another embodiment, the solid pharmaceutical composition includes a solid carrier, the solid carrier being formed of the acid-labile active ingredient and different combinations of hydrophilic surfactants, lipophilic additives and solubilizers. These and other embodiments, as well as preferred aspects thereof, are described in more detail below.

It should be appreciated that any of the components of the compositions of the present invention can be used as supplied commercially, or can be preprocessed by agglomeration, air suspension chilling, air suspension drying, balling, coacervation, comminution, compression, pelletization, cryopelletization, extrusion, granulation, homogenization, inclusion complexation, lyophilization, melting, mixing, molding, pan coating, solvent dehydration, sonication, spheronization, spray chilling, spray congealing, spray drying, or other processes known in the art. The various components can also be pre-coated or encapsulated. These various processes and coatings are described in more detail below.

1. Pharmaceutical Active Ingredients

The solid carrier of the present invention comprises a pharmaceutical active ingredient. The terms “active agent,” “pharmacologically active agent,” and “drug” are used interchangeably herein to refer to any chemical compound, complex or composition that has a beneficial biological effect, preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, isomers, fragments, analogs, and the like. When the terms “active agent,” “pharmacologically active agent” and “drug” are used, then, or when a particular active agent is specifically identified, it is to be understood that applicants intend to include the active agent per se as well as pharmaceutically acceptable, pharmacologically active salts, esters, amides, prodrugs, active metabolites, isomers, fragments, analogs, etc.

Any of the active agents may be administered in the form of a salt, ester, amide, prodrug, active metabolite, isomer, analog, fragment, or the like, provided that the salt, ester, amide, prodrug, active metabolite, isomer, analog or fragment, is pharmaceutically acceptable and pharmacologically active in the present context. Salts, esters, amides, prodrugs, metabolites, analogs, fragments, and other derivatives of the active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

The active ingredient can be solubilized, dispersed, or partially solubilized and dispersed, in the encapsulation coat. Alternatively, the active ingredient can be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, diagnostic agents, nutritional agents, and the like.

The active agents that may be administered using the compositions, systems and methods of the invention are not limited, as the invention enables the effective delivery of a wide variety of active agents. Therefore, the active agent administered may be selected from any of the various classes of such agents including, but not limited to, analgesic agents, anesthetic agents, anti-anginal agents, antiarthritic agents, anti-arrhythmic agents, antiasthmatic agents, antibacterial agents, anti-BPH agents, anticancer agents, anticholinergic agents, anticoagulants, anticonvulsants, antidepressants, antidiabetic agents, antidiarrheals, anti-epileptic agents, antifungal agents, anti-gout agents, antihelminthic agents, antihistamines, antihypertensive agents, antiinflammatory agents, anti-malarial agents, antimigraine agents, anti-muscarinic agents, antinauseants, antineoplastic agents, anti-obesity agents, anti-osteoporosis agents, antiparkinsonism agents, anti-protozoal agents, antipruritics, antipsychotic agents, antipyretics, antispasmodics, anti-thyroid agents, antitubercular agents, antiulcer agents, anti-urinary incontinence agents, antiviral agents, anxiolytics, appetite suppressants, attention deficit disorder (ADD) and attention deficit hyperactivity disorder (ADHD) drugs, calcium channel blockers, cardiac inotropic agents, beta-blockers, central nervous system stimulants, cognition enhancers, corticosteroids, COX-2 inhibitors, decongestants, diuretics, gastrointestinal agents, genetic materials, histamine receptor antagonists, hormonolytics, hypnotics, hypoglycemic agents, immunosuppressants, keratolytics, leukotriene inhibitors, lipid-regulating agents, macrolides, mitotic inhibitors, muscle relaxants, narcotic antagonists, neuroleptic agents, nicotine, nutritional oils, parasympatholytic agents, sedatives, sex hormones, sympathomimetic agents, tranquilizers, vasodilators, vitamins, and combinations thereof. Active agents that may be administered according to the invention also include nutrients, cosmeceuticals, diagnostic agents, and nutritional agents. Some agents, as will be appreciated by those of ordinary skill in the art, and as may be deduced from the discussion below, are encompassed by two or more of the aforementioned groups or other uses that it might be found appropriate for.

Among the various active agent categories, preferred classes of active agents for administration using the present method and formulations are gastrointestinal agents; lipid regulating agents, sex hormones, anti-hypertensive agents, anti-diabetic agents, anti-viral agents (including protease inhibitors), agents for treating neurodegenerative diseases (including anti-Parkinson's and anti-Alzheimer's), anxiolytics, sedatives, hypnotics, agents for treating headaches (including anti-migraine agents), neuroleptic drugs (including anti-depressants, anti-manics, anti-psychotics) and combinations of any of the foregoing:

gastrointestinal agents, such as alosetron, basalazide, bisacodyl, budesonide, cilansetron, cimetidine, cisapride, diphenoxylate, domperidone, esomeprazole, famotidine, granisetron, lafutidine, lansoprazole, leminoprazole, loperamide, merropenum, mesalazine, mesalamine, nitisonone, nizatidine, olsalazine, omeprazole, ondansetron, pantoprazole, palonosetron, pariprazole, rabeprazole sodium, ransoprazole, ranitidine, risperidone, sulphasalazine, and tegaserod;

neuroleptic drugs, including antidepressant drugs, anti-manic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, escitalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein anti-manic; and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, perospirone, pimozide, quetiapine, risperidone, sertindole, and ziprasidone;

agents for treating headaches, including anti-migraine agents, such as almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan;

agents to treat neurodegenerative diseases, including active agents for treating Alzheimer's disease such as akatinol, donezepil, donezepil hydrochloride, dronabinol, galanthamine, ipidracine, neotrofin, rasagiline, physostigmine, physostigmine salicylate, propentoffyline, quetiapine, rivastigmine, tacrine, tacrine hydrochloride, thalidomide, and xaliproden; active agents for treating Huntington's Disease, such as fluoxetine and carbamazepine; anti-Parkinson's drugs useful herein include amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride; and active agents for treating ALS such as the anti-spastic agents baclofen, diazemine, riluzole, and tizanidine; and active agents for multiple sclerosis such as glatiramer;

anxiolytics, sedatives, and hypnotics, such as alprazolam, amylobarbitone, barbitone, bentazepam, bromazepam, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threo-methylphenidate) diazepam, droperidol, ethinamate, flunanisone, flunitrazepam, triflupromazine, flupenthixol decanoate, fluphenazine, flurazepam, gabapentin, gaboxadol, γ-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, rohypnol, sertindole, siramesine, sulpiride, sunepitron, temazepam, thioridazine, triazolam, zaleplon, zolpidem, and zopiclone; and

lipid-regulating agents that are generally classified as hydrophobic include HMG CoA reductase inhibitors such as atorvastatin, simvastatin, fluvastatin, pravastatin, lovastatin, cerivastatin, rosuvastatin, and pitavastatin, as well as other lipid-lowering (“antihyperlipidemic”) agents such as bezafibrate, beclobrate, binifibrate, ciprofibrate, clinofibrate, clofibrate, clofibric acid, ezetimibe, etofibrate, fenofibrate, fenofibric acid, gemfibrozil, lifibrol, nicofibrate, pirifibrate, probucol, ronifibrate, simfibrate, and theofibrate. A particularly preferred lipid-regulating agent that may be administered using the methods and formulations of the invention is fenofibrate.

Preferred sex hormones for administration using according to the invention include progestins (progestogens), estrogens, and combinations thereof. Progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, and trimgestone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. In many contexts, e.g., in female contraception and in hormone replacement therapy (HRT), a combination of a progestin and estrogen is used, e.g., progesterone and 17 β-estradiol. For HRT, an androgenic agent may be advantageously included as well. Androgenic agents for this purpose include, for example, dehydroepiandrosterone (DHEA; also termed “prasterone”), sodium dehydroepiandrosterone sulfate, 4-dihydrotestosterone (DHT; also termed “stanolone”), and testosterone, and pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters.

Androgenic agents may also be administered for other purposes well known in the art. In addition to the androgenic agents enumerated above, other androgenic agents include, but are not limited to, androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, and dromostanolone propionate.

Other active agent categories suitable for administration using the present method and formulations include:

antihypertensive agents include, without limitation, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, imidapril, irbesartan, isradipine, lercardinipine, lisinopril, losartan, mibefradil, minoxidil, nebivolol, nicardipine, nifedipine, nimodipine, nisoldipine, olmesartan, omapatrilat, phenoxybenzamine, pindolol, prazosin, quinapril, reserpine, semotiadil, sitaxsentan, terazosin, telmisartan, trandolapril, and valsartan;

anti-diabetic agents include, by way of example, acetohexamide, chlorpropamide, ciglitazone, farglitazar, glibenclamide, gliclazide, glipizide, glucagon, glyburide, glymepiride, miglitol, pioglitazone, nateglinide, pimagedine, repaglinide, rosiglitazone, tolazamide, tolbutamide, triampterine, and troglitazone;

antiviral agents that can be delivered using the present methods and dosage forms include the antiherpes agents acyclovir, famciclovir, foscarnet, ganciclovir, idoxuridine, sorivudine, trifluridine, valacyclovir, and vidarabine, and other antiviral agents such as abacavir, amantadine, amprenavir, cidofovir, delviridine, didanosine, efavirenz, indinavir, interferon alpha, lamivudine, lobucavir, lopinavir, nelfinavir, nevirapine, oseltamivir, ribavirin, rimantadine, ritonavir, saquinavir, stavudine, tipranavir, valganciclovir, zanamivir, zalcitabine, and zidovudine; and other antiviral agents such as abacavir, indinavir, interferon alpha, nelfinavir, ribavirin, rimantadine, tipranavir, ursodeoxycholic acid, and valganciclovir;

anti-inflammatory agents and non-opioid analgesics, such as aloxiprin, amiprilose, auranofin, azapropazone, azathioprine, benorylate, boswellic acid, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine;

anti-angina agents, such as mibefradil, refludan, nalmefene, carvedilol, cromafiban, lamifiban, fasudil, ranolazine, tedisamil, nisoldipine, and tizanidine;

antihelminthics, such as albendazole, bephenium hydroxynaphthoate, cambendazole, dichlorophen, ivermectin, mebendazole, oxamniquine, oxfendazole, oxantel embonate, praziquantel, pyrantel embonate and thiabendazole;

anti-arrhythmic agents, such as amiodarone, disopyramide, flecainide acetate and quinidine sulfate;

anti-asthma agents, such as fudosteine, zileuton, zafirlukast, terbutaline sulfate, montelukast, pranlukast, levalbuterol, ramatroban, suplatast, and albuterol;

anti-bacterial agents, such as alatrofloxacin, azithromycin, baclofen, benethamine penicillin, cinoxacin, ciprofloxacin, cefoselis, ceffibuten, clarithromycin, clofazimine, cloxacillin, dalfopristine, demeclocycline, dirithromycin, doxycycline, ecenofloxacin, erythromycin, ethionamide, furazolidone, grepafloxacin, imipenem, levofloxacin, linezolid, lorefloxacin, moxifloxacin, nalidixic acid, nitrofurantoin, norfloxacin, ofloxacin, quinupritin, rifampicin, rifabutine, rifapentine, ritipenem, sparfloxacin, spiramycin, sulphabenzamide, sulphadoxine, sulphamerazine, sulphacetamide, sulphadiazine, sulphafurazole, sulphamethoxazole, sulphapyridine, tazobactum, tetracycline, tosufloxacin, trimethoprim, trovafloxacin, and vancomycin;

anti-cancer agents and immunosuppressants, such as alitretinoin, aminoglutethimide, amsacrine, anastrozole, azathioprine, bexarotene, bicalutamide, biricodar, bisantrene, busulfan, camptothecin, candoxatril, capecitabine, cisplatin, cytarabine, chlorambucil, cyclosporin, dacarbazine, decitabine, ellipticine, estramustine, etoposide, examorelin, examestane, fludarabine, gemcitabine, imatinib, irinotecan, lasofoxifene, letrozole, lomustine, melphalan, mercaptopurine, methotrexate, mitomycin, mitotane, mitoxantrone, mofetil, mycophenolate, nebivolol, nilutamide, oxaliplatin, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, venorelbine, and zacopride;

anti-coagulants and other agents for preventing and treating stroke, such agatroban, cilostazol, citicoline, clopidogrel, cromafiban, dexanabinol, dicoumarol, dipyridamole, nicoumalone, oprevelkin, ozagrel, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin;

anti-diabetics, such as acetohexamide, chlorpropamide, farglitazar, glibenclamide, gliclazide, glipizide, glimepiride, miglitol, nateglinide, pimagedine, pioglitazone, repaglinide, rosiglitazone, tolazamide, tolbutamide, troglitazone, and voglibose;

anti-epileptics, such as beclamide, carbamazepine, carbatrol, clobazam, clonazepam, divalproex sodium, ethotoin, felbamate, fosphenytoin, levetriacetam, lamotrigine, methoin, methsuximide, methylphenobarbitone, oxcarbazepine, paramethadione, phenacemide, phenobarbitone, phenytoin, phensuximide, primidone, sulthiame, tiagabine, tolcapone, topiramate, valproic acid, vigabatrin, and zonisamide;

anti-fungal agents, such as anidulafungin, amphotericin, butenafine, butoconazole nitrate, clotrimazole, econazole nitrate, fluconazole, flucytosine, griseofulvin, itraconazole, ketoconazole, liranaftate, miconazole, natamycin, nystatin, sulconazole nitrate, oxiconazole, terbinafine, terconazole, tioconazole and undecanoic acid;

anti-gout agents, such as allopurinol, probenecid and sulphinpyrazone;

antihistamines and allergy medications, such as acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine;

anti-malarials, such as amodiaquine, chloroquine, chlorproguanil, halofantrine, mefloquine, proguanil, pyrimethamine and quinine sulfate;

anti-muscarinic agents, such as atropine, benzhexol, beperiden, ethopropazine, hyoscyamine, mepenzolate bromide, oxyphencyclimine, scopolamine, and tropicamide;

anti-protozoal agents, such as atovaquone, benznidazole, clioquinol, decoquinate, diiodohydroxyquinoline, diloxanide furoate, dinitolmide, furazolidone, metronidazole, nimorazole, nitrofurazone, ornidazole and tinidazole;

anti-thyroid agents, such as carbimazole, paracalcitol, and propylthiouracil;

anti-tussives, such as benzonatate;

appetite suppressants, anti-obesity drugs and drugs for treatment of eating disorders, such as amphetamine, bromocriptine, dextroamphetamine, diethylpropion, ghrelin, lintitript, mazindol, methamphetamine, orlistat, phentermine, and topiramate;

cardiovascular drugs, including: angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides and cardiac inotropes such as amrinone, digoxin, digitoxin, enoximone, lanatoside C, medigoxin, and milrinone; calcium channel blockers such as verapamil, nifedipine, nicardipene, felodipine, isradipine, nimodipine, amlodipine and diltiazem; beta-blockers such as acebutolol, alprenolol, atenolol, labetalol, metoprolol, nadolol, oxyprenolol, pindolol, propafenone, propranolol, esmolol, sotalol, timolol, and acebutolol; antiarrhythmics such as mexiletine, moricizine, dofetilide, ibutilide, nesiritide, procainamide, quinidine, disopyramide, lidocaine, phenytoin, tocainide, mexiletine, flecainide, encainide, bretylium and amiodarone; cardioprotective agents such as dexrazoxane and leucovorin; vasodilators such as nitroglycerin; diuretic agents such as azetazolamide, amiloride, bendroflumethiazide, bumetanide, chlorothiazide, chlorthalidone, ethacrynic acid, furosemide, hydrochlorothiazide, metolazone, nesiritide, spironolactone, and triamterine; and miscellaneous cardiovascular drugs such as dopradil, midodrine, monatepil, monteplase, nexopamil, ranolazine, and pilsicainide;

corticosteroids, such as beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone;

cytoprotectant/antioxidants, such as dosmalfate, curcumin, edavarone;

erectile dysfunction drugs, such as apomorphine, phentolamine, and vardenafil;

keratolytics, such as such as acetretin, calcipotriene, calcifediol, calcitriol, cholecalciferol, ergocalciferol, etretinate, retinoids, targretin, and tazarotene;

lipid regulating agents, such as atorvastatin, bezafibrate, cerivastatin, ciprofibrate, clofibrate, ezetimibe, fenofibrate, fluvastatin, gemfibrozil, pitavastatin, pravastatin, probucol, rosuvastatin, and simvastatin;

muscle relaxants, such as cyclobenzaprine, dantrolene sodium, mexilitene, and tizanidine HCl;

nitrates and other anti-anginal agents, such as amyl nitrate, glyceryl trinitrate, isosorbide dinitrate, isosorbide mononitrate and pentaerythritol tetranitrate;

nutritional agents, such as calcitriol, carotenes, dihydrotachysterol, essential fatty acids, non-essential fatty acids, phytonadiol, vitamin A, vitamin B₂, vitamin D, vitamin E and vitamin K;

opioid analgesics, such as alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol; and

stimulants, including active agents for treating narcolepsy, attention deficit disorder (ADD) and attention deficit hyperactivity disorder (ADHD), such as amphetamine, dexamphetamine, dexfenfluramine, fenfluramine, mazindol, methylphenidate (including d-threo-methylphenidate, or “dexmethylphenidate,” as well as racemic d,1-threo-methylphenidate), modafinil, pemoline, and sibutramine.

Peptidyl drugs may also be delivered using the present methods and formulations. Peptidyl drugs include therapeutic peptides and proteins per se, whether naturally occurring, chemically synthesized, recombinantly produced, and/or produced by biochemical (e.g., enzymatic) fragmentation of larger molecules, and may contain the native sequence or an active fragment thereof. Specific peptidyl drugs include, without limitation, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B. insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP),and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage

colony-stimulating factor (GM-CSF), macrophage colony-stimulating factor, midkine (MD), and thymopoietin. Still other peptidyl drugs that can be advantageously delivered using the methodology and formulations of the present invention include endorphins (e.g., dermorphin, dynorphin, α-endorphin, β-endorphin, γ-endorphin, σ-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

Genetic material may also be delivered using the present methods and formulations, including, for example, nucleic acids, RNA, DNA, recombinant RNA, recombinant DNA, antisense RNA, antisense DNA, ribozymes, ribooligonucleotides, deoxyribonucleotides, antisense ribooligonucleotides, and antisense deoxyribooligonucleotides. Representative genes include those encoding for vascular endothelial growth factor, fibroblast growth factor, Bcl-2, cystic fibrosis transmembrane regulator, nerve growth factor, human growth factor, erythropoietin, tumor necrosis factor, and interleukin-2, as well as histocompatibility genes such as HLA-B7.

Other actives include: dutasetride for hair loss, granelix acetate for female infertility, incadronic acid for cancer or osteoporosis, pergolide for dopamine agonist activity, ritapentine, perenzepine, telenzepine, titanicene, limaprost, olopatidine, falecalcitriol, caldiribine, piapenum, farapenum, piracetam, tianeptine, adrafinil, vinpocetine, idebenone, oxiracetam, aniracetam, ketamine, ertapenum, cabergoline, acamprostate, nevibulol.

The active agent of the present invention can be hydrophobic, amphiphilic, or hydrophilic. The intrinsic water solubility of those active agents referred to as “hydrophobic” herein, i.e., the aqueous solubility of the active agent in electronically neutral, non-ionized form, is generally less than 1% by weight, and typically less than 0.1% or 0.01% by weight. Hydrophilic and amphiphilic active agents herein (which, unless otherwise indicated, are collectively referred to herein as “hydrophilic” active agents) have apparent water solubilities of at least 0.1% by weight, and typically at least 1% by weight. Both hydrophobic active agents and hydrophilic active agents may be selected from any of the active agent classes enumerated above.

It should be appreciated that the categorization of an active ingredient as hydrophobic or hydrophilic may change, depending upon the particular salts, isomers, analogs and derivatives used. For example, certain active agents indicated as hydrophobic may be readily converted to and commercially available in hydrophilic form, e.g., by ionizing a non-ionized active agent so as to form a pharmaceutically acceptable, pharmacologically active salt. Conversely, certain active agents indicated as hydrophilic may be readily converted to and commercially available in hydrophobic form, e.g., by neutralization, esterification, or the like. Thus, it should be understood that the above categorization of certain active agents as hydrophilic or hydrophobic is not intended to be limiting.

Specific, non-limiting examples of suitable hydrophobic active ingredients are: acetretin, acetyl coenzyme Q, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, esomeprazole, estradiol, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitrofurantoin, nizatidine, omeprazole, oprevelkin, oxaprozin, paclitaxel, pantoprazole, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Of course, salts, isomers and derivatives of the above-listed hydrophobic active ingredients may also be used, as well as mixtures thereof.

Among the above-listed hydrophobic active ingredients, preferred active ingredients include: acetretin, albendazole, albuterol, aminoglutethimide, amiodarone, amlodipine, amphetamine, amphotericin B, atorvastatin, atovaquone, azithromycin, baclofen, benzonatate, bicalutanide, busulfan, butenafine, calcifediol, calcipotriene, calcitriol, camptothecin, capsaicin, carbamezepine, carotenes, celecoxib, cerivastatin, chlorpheniramine, cholecaliferol, cimetidine, cinnarizine, ciprofloxacin, cisapride, cetirizine, clarithromycin, clemastine, clomiphene, codeine, coenzyme Q10, cyclosporin, danazol, dantrolene, dexchlorpheniramine, diclofenac, digoxin, dehydroepiandrosterone, dihydroergotamine, dihydrotachysterol, dirithromycin, donezepil, efavirenz, ergocalciferol, ergotamine, esomeprazole, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irinotecan, isotretinoin, itraconazole, ivermectin, ketoconazole, ketorolac, lamotrigine, lansoprazole, leflunomide, loperamide, loratadine, lovastatin, L-thryroxine, lutein, lycopene, mifepristone, mefloquine, megestrol acetate, methdone, methoxsalen, metronidazole, miconazole, midazolam, miglitol, mitoxantrone, medroxyprogesterone, montelukast, nabumetone, nalbuphine, naratriptan, nelfinavir, nilutanide, nitrofurantoin, nizatidine, omeprazole, oestradiol, oxaprozin, paclitaxel, pantoprazole, paracalcitol, pentazocine, pioglitazone, pizofetin, pravastatin, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sibutramine, sildenafil citrate, simvastatin, sirolimus, spironolactone, sumatriptan, tacrine, tacrolimus, tamoxifen, tamsulosin, targretin, tazarotene, teniposide, terbinafine, tetrahydrocannabinol, tiagabine, tizanidine, topiramate, topotecan, toremifene, tramadol, tretinoin, troglitazone, trovafloxacin, verteporfin, vigabatrin, vitamin A, vitamin D, vitamin E, vitamin K, zafirlukast, zileuton, ziprasidone, zolmitriptan, zolpidem, zopiclone, pharmaceutically acceptable salts, isomers and derivatives thereof, and mixtures thereof.

Particularly preferred hydrophobic active ingredients include: acetretin, albuterol, aminoglutethimide, amiodarone, amlodipine, amprenavir, atorvastatin, atovaquone, baclofen, benzonatate, bicalutanide, busulfan, calcifediol, calcipotriene, calcitriol, camptothecin, capsaicin, carbamezepine, carotenes, celecoxib, chlorpheniramine, cholecaliferol, cimetidine, cinnarizine, cisapride, cetirizine, clemastine, coenzyme Q10, cyclosporin, danazol, dantrolene, dexchlorpheniramine, diclofenac, dehydroepiandrosterone, dihydroergotamine, dihydrotachysterol, efavirenz, ergocalciferol, ergotamine, esomeprazole, essential fatty acid sources, etodolac, etoposide, famotidine, fenofibrate, fexofenadine, finasteride, fluconazole, flurbiprofen, fosphenytoin, frovatriptan, furazolidone, glibenclamide, glipizide, glyburide, glimepiride, ibuprofen, irinotecan, isotretinoin, itraconazole, ivermectin, ketoconazole, ketorolac, lamotrigine, lansoprazole, leflunomide, loperamide, loratadine, lovastatin, L-thryroxine, lutein, lycopene, medroxyprogesterone, mifepristone, megestrol acetate, methoxsalen, metronidazole, miconazole, miglitol, mitoxantrone, montelukast, nabumetone, naratriptan, nelfinavir, nilutanide, nitrofurantoin, nizatidine, omeprazole, oestradiol, oxaprozin, paclitaxel, pantoprazole, paracalcitol, pioglitazone, pizofetin, pranlukast, probucol, progesterone, pseudoephedrine, rabeprazole, raloxifene, rofecoxib, repaglinide, rifabutine, rifapentine, rimexolone, ritanovir, rizatriptan, rosiglitazone, saquinavir, sildenafil citrate, simvastatin, sirolimus, tacrolimus, tamoxifen, tamsulosin, targretin, tazarotene, teniposide, terbenafine, tetrahydrocannabinol, tiagabine, tizanidine, topiramate, topotecan, toremifene, tramadol, tretinoin, troglitazone, trovafloxacin, ubidecarenone, vigabatrin, vitamin A, vitamin D, vitamin E, vitamin K, zafirlukast, zileuton, ziprasidone, zolmitriptan, pharmaceutically acceptable salts, isomers and derivatives thereof, and mixtures thereof.

Specific, non-limiting examples of suitable hydrophilic active ingredients include: acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clodronate; colistimethate sodium; colistin sulfate; corticotropin; cosyntropin; coromlyn sodium; cytarabine; dalteparin sodium; danaparoid; desferrioxamine; denileukin diftitox; desmopressin; diatrizoate meglumine and diatrizoate sodium; dicyclomine; didanosine; dirithromycin; dopamine hydrochloride; dornase alpha; doxacurium chloride; doxorubicin; etidronate disodium; enalaprilat; enkephalin; enoxaparin; enoxaparin sodium; ephedrine; epinephrine; epoetin alpha; erythromycin; esmolol hydrochloride; factor IX; famciclovir; fludarabine; fluoxetine; foscarnet sodium; ganciclovir; granulocyte colony stimulating factor; granulocyte-macrophage stimulating factor; recombinant human growth hormones ; bovine growth hormone; gentamycin; glucagon; glycopyrolate; gonadotropin releasing hormone and synthetic analogs thereof; GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mepenzolate bromide; mesalamine; methenamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; phentolamine mesylate; phenylalanine; physostigmine salicylate; plague vaccine; piperacillin sodium; platelet derived growth factor; pneumococcal vaccine polyvalent; poliovirus vaccine (inactivated); poliovirus vaccine live (OPV); polymyxin B sulfate; pralidoxime chloride; pramlintide; pregabalin; propafenone; propantheline bromide; pyridostigmine bromide; rabies vaccine; residronate; ribavarin; rimantadine hydrochloride; rotavirus vaccine; salmeterol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecuronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof; and mixtures thereof.

Among the above-listed hydrophilic active ingredients, preferred active ingredients include acarbose; acyclovir; atracurium besylate; alendronate; alglucerase; amantadine hydrochloride; amphotericin B; antihemophilic factor (human); antihemophilic factor (porcine); antihemophilic factor (recombinant; azithromycin; calcitonin human; calcitonin salmon; capecitabine; cefazolin sodium; cefonicid sodium; cefoperazone; cefoxitin sodium; ceftizoxime; ceftriaxone; cefuroxime axetil; cephalexin; chorionic gonadotropin; cidofovir; cladribine; clindamycin and clindamycin derivatives; corticotropin; cosyntropin; coromlyn sodium; cytarabine; dalteparin sodium; danaparoid; desmopressin; didanosine; dirithromycin; etidronate disodium; enoxaparin sodium; epoetin alpha; factor IX; famciclovir; fludarabine; foscarnet sodium; ganciclovir; granulocyte colony stimulating factor; granulocyte-macrophage stimulating factor; growth hormones-recombinant human; growth hormone-bovine; gentamycin; glucagon; gonadotropin releasing hormone and synthetic analogs thereof; GnRH; gonadorelin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; lamivudine; leucovorin calcium; leuprolide acetate; lincomycin and lincomycin derivatives; metformin hydrochloride; nedocromil sodium; neostigmine bromide; neostigmine methyl sulfate; neurontin; octreotide acetate; olpadronate; pamidronate disodium; pancuronium bromide; pentamidine isethionate; pentagastrin; physostigmine salicylate; poliovirus vaccine live (OPV); pyridostigmine bromide; residronate; ribavarin; rimantadine hydrochloride; rotavirus vaccine; salmeterol xinafoate; somatostatin; spectinomycin; stavudine; streptokinase; ticarcillin; tiludronate; tissue type plasminogen activator; TNFR:Fc; TNK-tPA; trimetrexate gluconate; trospectinomycin; tumor necrosis factor; typhoid vaccine live; urokinase; vancomycin; valacyclovir; vasopressin and vasopressin derivatives; vinblastine; vincristine; vinorelbine; warfarin sodium; zalcitabine; zanamivir; zidovudine; pharmaceutically acceptable salts, isomers and derivatives thereof; and mixtures thereof.

Most preferred hydrophilic active ingredients include acarbose; alendronate; amantadine hydrochloride; azithromycin; calcitonin human; calcitonin salmon; ceftriaxone; cefuroxime axetil; chorionic gonadotropin; coromlyn sodium; dalteparin sodium; danaparoid; desmopressin; didanosine; etidronate disodium; enoxaparin sodium; epoetin alpha; factor IX; famciclovir; foscarnet sodium; ganciclovir; granulocyte colony stimulating factor; granulocyte-macrophage stimulating factor; growth hormones-recombinant human; growth hormone-bovine; glucagon; gonadotropin releasing hormone and synthetic analogs thereof, GnRH; gonadorelin; heparin sodium; indinavir sulfate; influenza virus vaccine; interleukin-2; interleukin-3; insulin-human; insulin lispro; insulin procine interferon alpha; interferon beta; leuprolide acetate; metformin hydrochloride; nedocromil sodium; neostigmine bromide; neostigmine methyl sulfate; neurontin; octreotide acetate; olpadronate; pamidronate disodium; residronate; rimantadine hydrochloride; salmeterol xinafoate; somatostatin; stavudine; ticarcillin; tiludronate; tissue type plasminogen activator; TNFR:Fc; TNK-tPA; tumor necrosis factor; typhoid vaccine live; vancomycin; valacyclovir; vasopressin and vasopressin derivatives; zalcitabine; zanamivir; zidovudine; pharmaceutically acceptable salts, isomers and derivatives thereof; and mixtures thereof.

In some embodiments of the present invention, acid-labile active ingredients are the preferred active ingredients for their particular chemical stability needs upon storage during the shelf life as well as upon administration inside the gastrointestinal tract. As used herein, the term “acid-labile” is intended to include those pharmaceutical active ingredients that are particularly prone to or susceptible to degrade in neutral, and in particular acid environments, such as is encountered with the acidic pH conditions of the stomach upon oral administration, as well as is encountered with acidic components that may be present in the pharmaceutical composition during the manufacturing process and/or storage. Many acid-labile drugs are compounds that are biologically inactive in their formulated state, but degradation or transformation in the acid stomach environment converts the drug into an biologically active form.

The degradation of acid-labile drugs such as lansoprazole, has often been characterized as resulting from the interaction of the active ingredient with the acidic residues of the enteric coating. Surprisingly, even in the absence of an enteric coating with any acidic residues, acid-labile drugs still degraded in pellet formulations. It was further discovered that degradation of the active ingredient in the pellet formulations increased when the pellets were stored in a humidified environment and directly exposed to the moisture. Therefore, it is concluded that the presence of moisture and the ability of the pellets to absorb moisture are critical to the stability of acid-labile drugs in the pellet formulation. It is also believed that acidity or acidic residues in excipients or actives may provide for conditions for degradation. Accordingly, this invention pertains to various compositions and methods by which the active ingredient is protected from absorbing moisture, thus minimizing degradation.

In the embodiments of the present invention which include acid-labile active ingredients, the acid-labile active ingredients suitable for use in the pharmaceutical compositions and methods of the present invention are not particularly limited, as the compositions are surprisingly capable of effectively delivering a wide variety of acid-labile active ingredients. For example, in one embodiment of the invention, the active ingredient is an acid-labile compound and is selected from the group consisting of substituted benzimidazoles, lactams, lactones, nucleosides and analogues thereof, nucleotides and analogues thereof, esters, amides, peptides, peptidomimetics, and proteins.

A preferred class of acid-labile active ingredients are benzimidazoles, particularly those useful as proton pump inhibitors such as esomeprazole, lansoprazole, omeprazole, pantoprazole, rabeprazole, pharmaceutically acceptable salts, isomers and derivatives thereof, and combinations thereof; and most preferably lansoprazole, pantoprazole, rabeprazole, pharmaceutically acceptable salts, isomers and derivatives thereof, and combinations thereof. Exemplary salts include the sodium, potassium, calcium and magnesium salts of the active ingredient. It is understood that reference made to an active ingredient, such as lansoprazole is intended to mean the active per se, as well as pharmaceutically acceptable salts, isomers and derivatives thereof.

The active ingredient can also be administered in combination with one or more *additional active ingredients. For example, proton-pump inhibitors can be administered with anti-microbial agents, antacids, gastric anti-secretory drugs, and antihistamines. Exemplary gastric anti-secretory drugs include histamine H₂ receptor antagonists such as cimetidine, famotidine, nizatidine, and ranitidine; and anti-ulcer drugs such aceglutamide aluminum, cadexomer iodine, cetraxate hydrochloride, enisoprost, isotiquimide, lavoltidine succinate, misoprostol, nizatidine, nolinium bromide, pifarnine, pirenzepine hydrochloride, remiprostol, roxatidine acetate hydrochloride, sucralfate, sucrosofate potassium, and tolimidone. Exemplary antihistamines include, for example, acrivastinet, antazoline phosphate, astemizole, azatadine, barmastine, bromodiphenhydramine, carbinoxamine, cetirizine, chlorpheniramine, cinnarizine, clemastine, closiramine, cycliramine, cyclizine, cyproheptadine, dexbrompheniramnine, dexchlorpheniramine, dimethindene, diphenhydramine, dorastine, doxylamine, ebastine, levocabastine, loratadine, mianserin, noberastine, orphenadrine, pyrabrom, pyrilamine, pyroxamnine, rocastine, rotoxamine, tazifylline, temelastine, terfenadine, tripelennamine, triprolidine, and zolamine, as well as pharmaceutically acceptable derivatives thereof. Exemplary anti-microbial agents and antacids are described below.

Any of the aforementioned active agents may also be administered in combination using the present formulations. Active agents administered in combination may be from the same therapeutic class (e.g., lipid-regulating agents or anticoagulants) or from different therapeutic classes (e.g., a lipid-regulating agent and an anticoagulant). Examples of particularly important drug combination products include, but are not limited to:

female contraceptive compositions containing both a progestogen and an estrogen;

female HRT compositions containing a progestogen, an estrogen, and an androgen;

combinations of lipid-regulating agents, e.g., (a) a fibrate and a statin, such as fenofibrate and atorvastatin, fenofibrate and simvastatin, fenofibrate and lovastatin, or fenofibrate and pravastatin; (b) a fibrate and nicotinic acid, such fenofibrate and niacin; and (c) a statin and a nicotinic acid, such as lovastatin and niacin;

combinations of a lipid-regulating agent and an antiviral agent, e.g., a fibrate and a protease inhibitor, such as fenofibrate and ritonavir;

combinations of a lipid-regulating agent and an anticoagulant, e.g., (a) a fibrate and a salicylate, such as fenofibrate and aspirin, (b) a fibrate and another anticoagulant, such as fenofibrate and clopidogrel, (c) a statin and a salicylate, such as simvastatin and aspirin, and (d) a statin and another anticoagulant such as pravastatin and clopidogrel;

combinations of a lipid-regulating agent and an antidiabetic agent, including (a) a fibrate and a insulin sensitizer such as a thiazolidinedione, e.g., fenofibrate and pioglitazone, or fenofibrate and rosiglitazone, (b) a fibrate and an insulin stimulant such as a sulfonylurea, e.g., fenofibrate and glimepiride, or fenofibrate and glipizide, a statin and insulin sensitizer such as a thiazolidinedione, e.g., lovastatin and pioglitazone, simvastatin and rosiglitazone, pravastatin and pioglitazone, or the like;

combinations of a lipid regulating agent and a cardiovascular drug, e.g., (a) a fibrate and a calcium channel blocker, such as fenofibrate and amlodipine, or fenofibrate and irbesartan, or (b) a statin and a calcium channel blocker, such as fosinopril and pravastatin;

combinations of anticoagulants, e.g., (a) a salicylate and a platelet receptor binding inhibitor, such as aspirin and clopidogrel, (b) a salicylate and a low molecular weight heparin, such as aspirin and dalteparin, and (c) a platelet receptor binding inhibitor and a low molecular weight heparin, such as clopidogrel and enoxaparin;

combinations of anti-diabetics, e.g., (a) an insulin sensitizer and an insulin stimulant, such as (i) a thiazolidinedione such as glitazone or pioglitazone and a sulfonylurea such as glimepiride, and (ii) a biguanide such as metformin and a meglitinide such as repaglinide, (b) an insulin sensitizer and an α-glucosidase inhibitor, such as metformin and acarbose, (c) an insulin stimulant and an α-glucosidase inhibitor, such as (i) a sulfonylurea such as glyburide combined with acarbose, (ii) acarbose and a meglitinide such as repaglinide, (iii) miglitol and a sulfonylurea such as glipizide, or (iv) acarbose and a thiazolidinedione such as pioglitazone;

combinations of cardiovascular drugs, such as combinations of ACE inhibitors, e.g., lisinopril and candesartan; a combination of an ACE inhibitor with a diuretic agent such as losartan and hydrochlorothiazide; a combination of a calcium channel blocker and a β-blocker such as nifedipine and atenolol; and a combination of a calcium channel blocker and an ACE inhibitor such as felodipine and ramipril;

combinations of an antihypertensive agent and an antidiabetic agent, such as an ACE inhibitor and a sulfonylurea, e.g., irbesartan and glipizide;

combinations of antihistamines and antiasthmatic agents, e.g., an antihistamine and a leukotriene receptor antagonist such as loratadine and zafirlukast, desloratidine and zafirlukast, and cetirazine and montelukast;

combinations of antiinflammatory agents and analgesics, e.g., a COX-2 inhibitor and a nonsteroidal antiinflammatory agent (NSAID) such as rofecoxib and naproxen, or a COX-2 inhibitor and a salicylate such as celecoxib and aspirin;

combinations of an anti-obesity drug and an antidiabetic agent, e.g., a lipase inhibitor such as orlistat in combination with metformin;

combinations of a lipid-regulating agent and a drug for treating coronary artery disease, e.g., fenofibrate and ezetimibe, or lovastatin and ezetimibe; and

other combinations, such as docetaxel and cisplatin, tirapazamine and cisplatin, metoclopramide and naproxen sodium, an opioid analgesic such as oxycodone and an anti-inflammatory agent, an agent for treating erectile dysfunction, such as alprostadil, with an antihypertensive/vasodilator such as prazosin.

In one embodiment of the invention, anti-microbial agents are included in the pharmaceutical composition, preferably with an acid-labile active ingredient. They can be admixed with the active ingredient, for example in the encapsulation coat. Alternately, the anti-microbial agents can be separate from the active ingredient, for example in the substrate. A preferred class of anti-microbial agents are those that are active against Helicobacter pylori These include, by way of illustration and not limitation, antibiotics, anti-mycobacterials, antiviral agents, bismuth salts, imidazole compounds, nitrofurans, quinolones, sulfonamides, systemic antifungal agents, other antimicrobial agents such as chloramphenicol, spectinomycin, polymyxin B (colistin), bacitracin, nitrofurantoin, methenamine mandelate, and methenamine hippurate; and combinations of any of the foregoing.

Exemplary antibiotics include, aminoglycosides (e.g., amikacin, clarithromycin, gentamicin, kanamycin, neomycin, netilmicin, paromomycin, streptomycin, tobramycin, spectinomycin); ampherlcols (e.g., chloramphenicol, thiamphenicol); beta.-lactam antibiotics including antistaphylococcal penicillins (e.g., cloxacillin, dicloxacillin, nafcillin, and oxacillin), penicillins (e.g., azidicillin, aziocillin, bacampicillin, benzylpenicillin, flucloxacillin, mecillinam, meziocillin, penicillin G, penicillin VK, phenoxymethylpenicillin, piperacillin, propicillin), extended spectrum penicillins (e.g., aminopenicillins such as ampicillin and amoxicillin, and the antipseudomonal penicillins such as carbenicillin), as well as aztreonam, loracarbef, and meropenem); carbapenems (e.g., aztreonam, imipenem, loracarbef, meropenem, panipenem); cephalosporins (e.g., cefaclor, cefadroxil, cefalexin, cefamandol, cefazolin, cefepime, cefetamet, cefixime, cefmenoxim, cefodizime cefoperazon, cefotaxime, cefotetan, cefotiam, cefoxitin, cefpodoxim, cefsulodin, ceftaxim, ceftazidime, ceftibuten, ceftizoxim, ceftriaxone, cefuraxime, cefuroxim, cephalexin, cephalothin); glycopeptides (e.g., teicoplanin); gyrase inhibitors (e.g., cinoxacin, ciprofloxacin, enoxacin, fieroxacin, nalidixie acid, norfloxacin, ofloxacin, perfloxacin, pipemidic acid); macrolides (e.g., azithromycin, clarithromycin, clindamycin, erythromycin, lincomycin, spiramycin, rifampicin, roxithromycin); polypeptides (e.g., bacitracin, collstin, polymyxin B, teioplanin, vancomycin); streptogramin antibiotics (e.g., dalfopristin, quinupristin); tetracyclines (e.g., chlortetracycline, demeclocycline, doxycycline, methacycline, minocycline, oxytetracycline, rolitetracycline, tetracycline); as well as combinations thereof. Exemplary combinations include a bismuth salt and/or tetracycline with metronidazole, amoxicillin or clarithromycin with metronidazole, and amoxicillin with clarithromycin.

Exemplary anti-mycobacterial agents include aminosalicylic, cycloserine, ethambutol, ethionamide, isoniazid, pyrazinamide, rifabutin, and rifampin.

Exemplary antiviral agents include acyclovir, amantadine, didanosine, famcicylovir, ganciclovir, idoxuridine, interferon alpha, ribavirin, rimantadine, stavudine, sorivudine, trifluridine, valacyclovir, vidarabine, zalcitabine, and zidovudine.

Exemplary bismuth salts include α-D-glucopyranoside bismuth complex, β-D-fructofuranosyl-oktakis (hydrogen sulfate) bismuth complex, bismuth aluminate, bismuth citrate, bismuth polyhydroxy complexes, bismuth polysulfate complexes, bismuth salicylate, bismuth subacetate, bismuth subcarbonate, bismuth subcitrate, bismuth subgallate, bismuth subsalicylate, bismuth subnitrate, bismuth tartrate, colloidal bismuth subcitrate, L-dihydro ascorbyl-tetrakis (hydrogen sulfate) bismuth complex, and tripotassium dicitrato bismuthate.

Exemplary imidazole compounds include metronidazole, miconazole, nimorazole, and tinidazole. Exemplary nitrofurans include furazolidone, nitrofurantoin, and nitrofurazone. Exemplary quinolones include ciprofloxacin, nalidixic acid, and ofloxacin. Exemplary sulfonamides include sulfabenzamide, sulfacetamide, sulfadiazine, sulfadoxine, sulfamerazine, sulfamethazine, sulfamethizole, and sulfamethoxazole.

Exemplary systemic antifungal agents include amphotericin B, ketoconazole, fluconazole, and itraconazole.

Preferred anti-microbial agents include, amoxicillin, clarithromycin, erythromycin, metronidazole, tetracycline, and combinations thereof.

In another embodiment of the invention, an antacid agent is included in the pharmaceutical composition, preferably with an acid-labile active ingredient. They can be admixed with the active ingredient, for example in the encapsulation coat. Alternately, the antacid agent can be separate from the active ingredient, for example in the substrate. Exemplary antacids include, aluminum hydroxide, calcium carbonate, magnesium hydroxide, sodium carbonate, and combinations thereof.

2. Surfactants

Various embodiments of the invention, as described in more detail below, include a hydrophilic surfactant. Hydrophilic surfactants can be used to provide any of several advantageous characteristics to the compositions, including: increased solubility of the active ingredient in the solid carrier; improved dissolution of the active ingredient; improved solubilization of the active ingredient upon dissolution; enhanced absorption and/or bioavailability of the active ingredient, particularly a hydrophilic active ingredient; and improved stability, both physical and chemical, of the active ingredient. The hydrophilic surfactant can be a single hydrophilic surfactant or a mixture of hydrophilic surfactants, and can be ionic or non-ionic.

Likewise, various embodiments of the invention include a lipophilic additive, which can be a lipophilic surfactant, including a mixture of lipophilic surfactants, a triglyceride, or a mixture thereof. The lipophilic surfactant can provide any of the advantageous characteristics listed above for hydrophilic surfactants, as well as further enhancing the function of the surfactants. These various embodiments are described in more detail below. For convenience, the surfactants are described in this section, and the triglycerides in the section that follows.

As is well known in the art, the terms “hydrophilic” and “lipophilic” are relative terms. To function as a surfactant, a compound must necessarily include polar or charged hydrophilic moieties as well as non-polar hydrophobic (lipophilic) moieties; i.e., a surfactant compound must be amphiphilic. An empirical parameter commonly used to characterize the relative hydrophilicity and lipophilicity of non-ionic amphiphilic compounds is the hydrophilic-lipophilic balance (the “HLB” value). Surfactants with lower HLB values are more lipophilic, and have greater solubility in oils, whereas surfactants with higher HLB values are more hydrophilic, and have greater solubility in aqueous solutions.

Using HLB values as a rough guide, hydrophilic surfactants are generally considered to be those compounds having an HLB value greater than about 10, as well as anionic, cationic, or zwitterionic compounds for which the HLB scale is not generally applicable. Similarly, lipophilic surfactants are compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or lipophilicity for use in the present invention, as described herein.

Surfactants can be any surfactant suitable for use in pharmaceutical compositions. Suitable surfactants can be anionic, cationic, zwitterionic or non-ionic. Such surfactants can be grouped into the following general chemical classes detailed in the Tables herein. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the present inventors, is more reliable.

It should be emphasized that the invention is not limited to the surfactants in the Tables, which show representative, but not exclusive, lists of available surfactants. In addition, refined, distilled or fractionated surfactants, purified fractions thereof, or re-esterified fractions, are also within the scope of the invention, although not specifically listed in the Tables.

2.1. Polyethoxylated Fatty Acids

Although polyethylene glycol (PEG) itself does not function as a surfactant, a variety of PEG-fatty acid esters have useful surfactant properties. Among the PEG-fatty acid monoesters, esters of lauric acid, oleic acid, and stearic acid are especially useful. Examples of polyethoxylated fatty acid monoester surfactants commercially available are shown in Table 1, with exemplary preferred hydrophilic surfactants including PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate.

TABLE 1
PEG-Fatty Acid Monoester Surfactants

| COMPOUND | COMMERCIAL PRODUCT (Supplier) | HLB |
|---|---|---|
| PEG 4-100 monolaurate | Crodet L series (Croda) | >9 |
| PEG 4-100 monooleate | Crodet O series (Croda) | >8 |
| PEG 4-100 monostearate | Crodet S series (Croda), Myrj Series (Atlas/ICI) | >6 |
| PEG 400 distearate | Cithrol 4 DS series (Croda) | >10 |
| PEG 100,200,300 | Cithrol ML series (Croda) | >10 |
| monolaurate | | |
| PEG 100,200,300 | Cithrol MO series (Croda) | >10 |
| monooleate | | |
| PEG 400 dioleate | Cithrol 4 DO series (Croda) | >10 |
| PEG 400-1000 | Cithrol MS series (Croda) | >10 |
| monostearate | | |
| PEG-1 stearate | Nikkol MYS-1EX (Nikko), Coster K1 (Condea) | 2 |
| PEG-2 stearate | Nikkol MYS-2 (Nikko) | 4 |
| PEG-2 oleate | Nikkol MYO-2 (Nikko) | 4.5 |
| PEG-4 laurate | Mapeg ® 200 ML (PPG), Kessco ® PEG 200 ML (Stepan), | 9.3 |
| | LIPOPEG 2 L (Lipo Chem.) | |
| PEG-4 oleate | Mapeg ® 200 MO (PPG), Kessco ® PEG 200 MO (Stepan), | 8.3 |
| PEG-4 stearate | Kessco ® PEG 200 MS (Stepan), Hodag 20 S (Calgene), | 6.5 |
| | Nikkol MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), Kessco ® PEG 300 MO | 8.5 |
| | Stepan), Nikkol MYO-6 (Nikko), Emulgante A6 | |
| | (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), LIPOPEG 4 DL (Lipo Chem.) | 13 |
| PEG-8 oleate | Mapeg ® 400 MO (PPG), Emulgante A8 (Condea) | 12 |
| PEG-8 stearate | Mapeg ® 400 MS (PPG), Myrj 45 | 12 |
| PEG-9 oleate | Emulgante A9 (Condea) | >10 |
| PEG-9 stearate | Cremophor S9 (BASF) | >10 |
| PEG-10 laurate | Nikkol MYL-10 (Nikko), Lauridac 10 (Croda) | 13 |
| PEG-10 oleate | Nikkol MYO-10 (Nikko) | 11 |
| PEG-10 stearate | Nikkol MYS-10 (Nikko), Coster K100 (Condea) | 11 |
| PEG-12 laurate | Kessco ® PEG 600 ML (Stepan) | 15 |
| PEG-12 oleate | Kessco ® PEG 600 MO (Stepan) | 14 |
| PEG-12 ricinoleate | (CAS # 9004-97-1) | >10 |
| PEG-12 stearate | Mapeg ® 600 MS (PPG), Kessco ® PEG 600 MS (Stepan) | 14 |
| PEG-15 stearate | Nikkol TMGS-15 (Nikko), Koster K15 (Condea) | 14 |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), Kessco ® PEG 1000 MS Stepan), | 16 |
| | Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 (Henkel), Nikkol MYS-40 (Nikko) | >10 |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet O-100 (Croda) | 18.8 |
| PEG-100 stearate | Myrj 59, Arlacel 165 (ICI) | 19 |
| PEG-200 oleate | Albunol 200 MO (Taiwan Surf.) | >10 |
| PEG-400 oleate | LACTOMUL (Henkel), Albunol 400 MO (Taiwan Surf.) | >10 |
| PEG-600 oleate | Albunol 600 MO (Taiwan Surf.) | >10 |

2.2 PEG-Fatty Acid Diesters

Polyethylene glycol (PEG) fatty acid diesters are also suitable for use as surfactants in the compositions of the present invention. Representative PEG-fatty acid diesters are shown in Table 2, with exemplary preferred hydrophilic surfactants including PEG-20 dilaurate, PEG-20 dioleate, PEG-20 distearate, PEG-32 dilaurate, and PEG-32 dioleate.

TABLE 2
PEG-Fatty Acid Diester Surfactants

| COMPOUND | COMMERCIAL PRODUCT (Supplier) | HLB |
|---|---|---|
| PEG-4 dilaurate | Mapeg ® 200 DL (PPG), Kessco ® PEG 200 DL (Stepan), | 7 |
| | LIPOPEG 2-DL (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), Kessco ® PEG 400 DL (Stepan), | 11 |
| | LIPOPEG 4 DL (Lipo Chem.) | |
| PEG-8 dioleate | Mapeg ® 400 DO (PPG), Kessco ® PEG 400 DO (Stepan), | 8.8 |
| | LIPOPEG 4 O (Lipo Chem.) | |
| PEG-8 distearate | Mapeg ® 400 DS (PPG), CDS 400 (Nikkol) | 11 |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), Kessco ® 600 DO (Stepan) | 10 |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4 DO series (Croda) | >10 |
| PEG-400 distearate | Cithrol 4 DS series (Croda) | >10 |

2.3 PEG-Fatty Acid Mono- and Di-ester Mixtures

In general, mixtures of surfactants are also useful in the present invention, including mixtures of two or more commercial surfactant products. Several PEG-fatty acid esters are marketed commercially as mixtures or mono- and diesters. Representative surfactant mixtures are shown in Table 3.

TABLE 3
PEG-Fatty Acid Mono- and Diester Mixtures

| | COMMERCIAL |
|---|---|
| COMPOUND | PRODUCT (Supplier) |
| PEG 4-150 mono, dilaurate | Kessco ® PEG 200-6000 mono, |
| | dilaurate (Stepan) |
| PEG 4-150 mono, dioleate | Kessco ® PEG 200-6000 mono, |
| | dioleate (Stepan) |
| PEG 4-150 mono, distearate | Kessco ® 200-6000 mono, |
| | distearate (Stepan) |

2.4 Polyethylene Glycol Glycerol Fatty Acid Esters

Suitable PEG glycerol fatty acid esters are shown in Table 4, with preferred hydrophilic surfactants including PEG-20 glyceryl laurate, PEG-30 glyceryl laurate, PEG-40 glyceryl laurate, PEG-20 glyceryl oleate, and PEG-30 glyceryl oleate.

TABLE 4
PEG Glycerol Fatty Acid Esters

| COMPOUND | COMMERCIAL PRODUCT (Supplier) | HLB |
|---|---|---|
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), Aldo ® | 13 |
| | MS-20 KFG (Lonza) | |
| PEG-20 glyceryl oleate | Tagat ® O (Goldschmidt) | >10 |
| PEG-30 glyceryl oleate | Tagat ® O2 (Goldschmidt) | >10 |

2.5. Alcohol—Oil Transesterification Products

A large number of surfactants of different degrees of lipophilicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas®-35), PEG-40 hydrogenated castor oil (Cremophor® RH 40), PEG-25 trioleate (TAGAT® TO), PEG-60 corn glycerides (Crovol® M70), PEG-60 almond oil (Crovol® A70), PEG-40 palm kernel oil (Crovol® PK70), PEG-50 castor oil (Emalex® C-50), PEG-50 hydrogenated castor oil (Emalex® HC-50), PEG-8 caprylic/capric glycerides (Labrasol®), and PEG-6 caprylic/capric glycerides (Softigen® 767). Preferred lipophilic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol® M40), and PEG-20 almond glycerides (Crovol® A40). The latter two surfactants are reported to have HLB values of 10, which is generally considered to be the approximate border line between hydrophilic and lipophilic surfactants. For purposes of the present invention, these two surfactants are considered to be lipophilic. Representative surfactants of this class suitable for use in the present invention are shown in Table 5.

TABLE 5
Transesterification Products of Oils and Alcohols

| COMPOUND | COMMERCIAL PRODUCT (Supplier) | HLB |
|---|---|---|
| PEG-3 castor oil | Nikkol CO-3 (Nikko) | 3 |
| PEG-5, 9, and 16 castor oil | ACCONON CA series (ABITEC) | 6-7 |
| PEG-20 castor oil | Emalex C-20 (Nihon Emulsion), Nikkol CO-20 TX | 11 |
| | (Nikko) | |
| PEG-23 castor oil | Emulgante EL23 | >10 |
| PEG-30 castor oil | Emalex C-30 (Nihon Emulsion), Alkamuls ® EL 620 | 11 |
| | (Rhone-Poulenc), Incrocas 30 (Croda) | |
| PEG-35 castor oil | Cremophor EL and EL-P (BASF), Emulphor EL, | |
| | Incrocas-35 (Croda), Emulgin RO 35 (Henkel) | |
| PEG-38 castor oil | Emulgante EL 65 (Condea) | |
| PEG-40 castor oil | Emalex C-40 (Nihon Emulsion), Alkamuls ® EL 719 | 13 |
| | (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated castor oil | Nikkol HCO-5 (Nikko) | 6 |
| PEG-7 hydrogenated castor oil | Simusol ® 989 (Seppic), Cremophor WO7 (BASF) | 6 |
| PEG-10 hydrogenated castor oil | Nikkol HCO-10 (Nikko) | 6.5 |
| PEG-20 hydrogenated castor oil | Nikkol HCO-20 (Nikko) | 11 |
| PEG-25 hydrogenated castor oil | Simulsol ® 1292 (Seppic), Cerex ELS 250 (Auschem | 11 |
| | SpA) | |
| PEG-30 hydrogenated castor oil | Nikkol HCO-30 (Nikko) | 11 |
| PEG-40 hydrogenated castor oil | Cremophor RH 40 (BASF), Croduret (Croda), | 13 |
| | Emulgin HRE (Henkel) | |
| PEG-45 hydrogenated castor oil | Cerex ELS 450 (Auschem Spa) | 14 |
| PEG-50 hydrogenated castor oil | Emalex HC-50 (Nihon Emulsion) | 14 |
| PEG-60 hydrogenated castor oil | Nikkol HCO-60 (Nikko); Cremophor RH 60 (BASF) | 15 |
| PEG-80 hydrogenated castor oil | Nikkol HCO-80 (Nikko) | 15 |
| PEG-100 hydrogenated castor oil | Nikkol HCO-100 (Nikko) | 17 |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel oil | Labrafil ® M 1944 CS (Gattefosse) | 4 |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated palm kernel | Labrafil ® M 2130 BS (Gattefosse) | 4 |
| oil | | |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn glycerides | Crovol M40 (Croda) | 10 |
| PEG-20 almond glycerides | Crovol A40 (Croda) | 10 |
| PEG-25 trioleate | TAGAT ® TO (Goldschmidt) | 11 |
| PEG-40 palm kernel oil | Crovol PK-70 | >10 |
| PEG-60 corn glycerides | Crovol M70 (Croda) | 15 |
| PEG-60 almond glycerides | Crovol A70 (Croda) | 15 |
| PEG-4 caprylic/capric triglyceride | Labrafac ® Hydro (Gattefosse), | 4-5 |
| PEG-8 caprylic/capric glycerides | Labrasol (Gattefosse), Labrafac CM 10 (Gattefosse) | >10 |
| PEG-6 caprylic/capric glycerides | SOFTIGEN ® 767 (Huls), Glycerox 767 (Croda) | 19 |
| Lauroyl macrogol-32 glyceride | GELUCIRE 44/14 (Gattefosse) | 14 |
| Stearoyl macrogol glyceride | GELUCIRE 50/13 (Gattefosse) | 13 |
| Mono, di, tri, tetra esters of | SorbitoGlyceride (Gattefosse) | <10 |
| vegetable oils and sorbitol | | |
| Pentaerythrityl tetraisostearate | Crodamol PTIS (Croda) | <10 |
| Pentaerythrityl distearate | Albunol DS (Taiwan Surf.) | <10 |
| Pentaerythrityl tetraoleate | Liponate PO-4 (Lipo Chem.) | <10 |
| Pentaerythrityl tetrastearate | Liponate PS-4 (Lipo Chem.) | <10 |
| Pentaerythrityl | Liponate PE-810 (Lipo Chem.), Crodamol PTC | <10 |
| tetracaprylate/tetracaprate | (Croda) | |
| Pentaerythrityl tetraoctanoate | Nikkol Pentarate 408 (Nikko) | |

Also included as oils in this category of surfactants are oil-soluble vitamin substances. The oil-soluble vitamin substances include vitamins A, D, E, K, and isomers, analogues, and derivatives thereof. The derivatives include organic acid esters of these oil-soluble vitamin substances, such as the esters of vitamin E or vitamin A with succinic acid. Thus, derivatives of these vitamins, such as tocopheryl PEG-1000 succinate (Vitamin E TPGS, available from Eastman) and other tocopheryl PEG succinate derivatives with various molecular weights of the PEG moiety, such as PEG 100-8000, are also suitable surfactants.

2.6. Polyglycerized Fatty Acids

Polyglycerol esters of fatty acids are also suitable surfactants for the present invention. Among the polyglyceryl fatty acid esters, preferred lipophilic surfactants include polyglyceryl oleate (Plurol Oleique®), polyglyceryl-2 dioleate (Nikkol DGDO), and polyglyceryl-10 trioleate. Preferred hydrophilic surfactants include polyglyceryl-10 laurate (Nikkol Decaglyn 1-L), polyglyceryl-10 oleate (Nikkol Decaglyn 1-O), and polyglyceryl-10 mono dioleate (Caprol® PEG 860). Polyglyceryl polyricinoleates (Polymuls) are also preferred hydrophilic and hydrophobic surfactants. Examples of suitable polyglyceryl esters are shown in Table 6.

TABLE 6
Polyglycerized Fatty Acids

| | COMMERCIAL | |
|---|---|---|
| COMPOUND | PRODUCT (Supplier) | HLB |
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 isostearate | Nikkol DGMIS (Nikko) | 5-7 |
| Polyglyceryl-3 oleate | Caprol ® 3GO (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), Nikkol | 9 |
| | Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 ricinoleate | Nikkol Hexaglyn PR-15 (Nikko) | >8 |
| Polyglyceryl-10 linoleate | Nikkol Decaglyn 1-LN (Nikko) | 12 |
| Polyglyceryl-6 pentaoleate | Nikkol Hexaglyn 5-O (Nikko) | <10 |
| Polyglyceryl-3 dioleate | Cremophor GO32 (BASF) | <10 |
| Polyglyceryl-3 distearate | Cremophor GS32 (BASF) | <10 |
| Polyglyceryl-4 pentaoleate | Nikkol Tetraglyn 5-O (Nikko) | <10 |
| Polyglyceryl-6 dioleate | Caprol ® 6G20 (ABITEC); | 8.5 |
| | Hodag PGO-62 (Calgene), | |
| | PLUROL OLEIQUE CC | |
| | 497 (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 pentaoleate | Nikkol Decaglyn 5-O (Nikko) | 3.5 |
| Polyglyceryl-10 septaoleate | Nikkol Decaglyn 7-O (Nikko) | 3 |
| Polyglyceryl-10 tetraoleate | Caprol ® 10G4O (ABITEC); | 6.2 |
| | Hodag PGO-62 (CALGENE), | |
| | Drewpol 10-4-O (Stepan) | |
| Polyglyceryl-10 | Nikkol Decaglyn 10-IS (Nikko) | <10 |
| decaisostearate | | |
| Polyglyceryl-101 decaoleate | Drewpol 10-10-O (Stepan), | 3.5 |
| | Caprol 10G10O (ABITEC), | |
| | Nikkol Decaglyn 10-O | |
| Polyglyceryl-10 mono, dioleate | Caprol ® PGE 860 (ABITEC) | 11 |
| Polyglyceryl polyricinoleate | Polymuls (Henkel) | 3-20 |

2.7. Propylene Glycol Fatty Acid Esters

Esters of propylene glycol and fatty acids are suitable surfactants for use in the present invention. In this surfactant class, preferred lipophilic surfactants include propylene glycol monocaprylate (Capryol® 90), propylene glycol monolaurate (Lauroglycol FCC), propylene glycol ricinoleate (Propymuls®), propylene glycol monooleate (Myverol® P-O6), propylene glycol dicaprylate/dicaprate (Captex® 200; Miglylol® 840), and propylene glycol dioctanoate (Captex® 800). Examples of surfactants of this class are given in Table 7.

TABLE 7
Propylene Glycol Fatty Acid Esters

| | COMMERCIAL | |
|---|---|---|
| COMPOUND | PRODUCT (Supplier) | HLB |
| Propylene glycol | Capryol 90 (Gattefosse), Nikkol | <10 |
| monocaprylate | Sefsol 218 (Nikko) | |
| Propylene glycol | Lauroglycol 90 (Gattefosse), | <10 |
| monolaurate | Lauroglycol FCC (Gattefosse) | |
| Propylene glycol oleate | Lutrol OP2000 (BASF) | <10 |
| Propylene glycol myristate | Mirpyl | <10 |
| Propylene glycol | ADM PGME-03 (ADM), | 3-4 |
| monostearate | LIPO PGMS (Lipo Chem.), | |
| | Aldo ® PGHMS (Lonza) | |
| Propylene glycol hydroxy | | <10 |
| stearate | | |
| Propylene glycol ricinoleate | PROPYMULS (Henkel) | <10 |
| Propylene glycol isostearate | | <10 |
| Propylene glycol monooleate | Myverol P-O6 (Eastman) | <10 |
| Propylene glycol | Captex ® 200 (ABITEC), | >6 |
| dicaprylate/dicaprate | Miglyol ® 840 (Huls), | |
| | Neobee ® M- | |
| | 20 (Stepan) | |
| Pro pylene glycol dioctanoate | Captex ® 800 (ABITEC) | >6 |
| Propylene glycol | LABRAFAC PG (Gattefosse) | >6 |
| caprylate/caprate | | |
| Propylene glycol dilaurate | | >6 |
| Propylene glycol distearate | Kessco ® PGDS (Stepan) | >6 |
| Propylene glycol dicaprylate | Nikkol Sefsol 228 (Nikko) | >6 |
| Propylene glycol dicaprate | Nikkol PDD (Nikko) | >6 |

2.8. Mixtures of Propylene Glycol Esters—Glycerol Esters

In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel® 186). Examples of these surfactants are shown in Table 8.

TABLE 8
Glycerol/Propylene Glycol Fatty Acid Esters

| COMPOUND | COMMERCIAL PRODUCT (Supplier) | HLB |
|---|---|---|
| Oleic | ATMOS 300, ARLACEL 186 (ICI) | 3-4 |
| Stearic | ATMOS 150 | 3-4 |

2.9. Mono- and Diglycerides

A particularly important class of surfactants is the class of mono- and diglycerides. These surfactants are generally lipophilic. Preferred lipophilic surfactants in this class of compounds include glyceryl monooleate (Peceol), glyceryl ricinoleate, glyceryl laurate, glyceryl dilaurate (Capmul® GDL), glyceryl dioleate (Capmul® GDO), glyceryl mono- and di-oleate (Capmul® GMO-K), glyceryl caprylate/caprate (Capmul® MCM), caprylic acid mono- and di-glycerides (Imwitor® 988), and mono- and diacetylated monoglycerides (Myvacet® 9-45). Examples of these surfactants are given in Table 9.

TABLE 9
Mono- and Diglyceride Surfactants

| COMPOUND | COMMERCIAL PRODUCT (Supplier) | HLB |
|---|---|---|
| Monopalmitolein (C16:1) | (Larodan) | <10 |
| Monoelaidin (C18:1) | (Larodan) | <10 |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl monomyristate | Nikkol MGM (Nikko) | 3-4 |
| (C14) | | |
| Glyceryl monooleate (C18:1) | PECEOL (Gattefosse), Hodag GMO-D, Nikkol MGO | 3-4 |
| | (Nikko) | |
| Glyceryl monooleate | RYLO series (Danisco), DIMODAN series (Danisco), | 3-4 |
| | EMULDAN (Danisco), ALDO ® MO FG (Lonza), | |
| | Kessco GMO (Stepan), MONOMULS ® series | |
| | (Henkel), TEGIN O, DREWMULSE GMO (Stepan), | |
| | Atlas G-695 (ICI), GMOrphic 80 (Eastman), ADM | |
| | DMG-40, 70, and 100 (ADM), Myverol (Eastman) | |
| Glycerol | OLICINE (Gattefosse) | 3-4 |
| monooleate/linoleate | | |
| Glycerol monolinoleate | Maisine (Gattefosse), Myverol 18-92, Myverol 18-06 | 3-4 |
| | (Eastman) | |
| Glyceryl ricinoleate | Softigen ® 701 (Huls), HODAG GMR-D (Calgene), | 6 |
| | ALDO ® MR (Lonza) | |
| Glyceryl monolaurate | ALDO ® MLD (Lonza), Hodag GML (Calgene) | 6.8 |
| Glycerol monopalmitate | Emalex GMS-P (Nihon) | 4 |
| Glycerol monostearate | Capmul ® GMS (ABITEC), Myvaplex (Eastman), Imwitor ® | 5-9 |
| | 191 (Huls), CUTINA ® GMS, Aldo ® MS (Lonza), | |
| | Nikkol MGS series (Nikko) | |
| Glyceryl mono-,dioleate | Capmul ® GMO-K (ABITEC) | <10 |
| Glyceryl palmitic/stearic | CUTINA MD-A, ESTAGEL-G18 | <10 |
| Glyceryl acetate | Lamegin ® EE (Grunau GmbH) | <10 |
| Glyceryl laurate | Imwitor ® 312 (Huls), Monomuls ® 90-45 (Grunau GmbH), | 4 |
| | Aldo ® MLD (Lonza) | |
| Glyceryl | Imwitor ® 375 (Huls) | <10 |
| citrate/lactate/oleate/ | | |
| linoleate | | |
| Glyceryl caprylate | Imwitor ® 308 (Huls), Capmul ® MCMC8 (ABITEC) | 5-6 |
| Glyceryl caprylate/caprate | Capmul ® MCM (ABITEC) | 5-6 |
| Caprylic acid | Imwitor ® 988 (Huls) | 5-6 |
| mono/diglycerides | | |
| Caprylic/capric glycerides | Imwitor ® 742 (Huls) | <10 |
| Mono-and diacetylated | Myvacet ® 9-45, Myvacet ® 9-40, Myvacet ® 9-08 | 3.8-4 |
| monoglycerides | (Eastman), Lamegin ® (Grunau) | |
| Glyceryl monostearate | Aldo ® MS, Arlacel 129 (ICI), LIPO GMS (Lipo Chem.), | 4.4 |
| | Imwitor ® 191 (Huls), Myvaplex (Eastman) | |
| Lactic acid esters of | LAMEGIN GLP (Henkel) | <10 |
| mono,diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | <10 |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of fatty acids | GELUCIRE 39/01 (Gattefosse), GELUCIRE 43/01 | 1 |
| | (Gattefosse) | |
| | GELUCIRE 37/06 (Gattefosse) | 6 |
| Dipalmitolein (C16:1) | (Larodan) | <10 |
| 1,2 and 1,3-diolein (C18:1) | (Larodan) | <10 |
| Dielaidin (C18:1) | (Larodan) | <10 |
| Dilinolein (C18:2) | (Larodan) | <10 |

2.10. Sterol and Sterol Derivatives

Sterols and derivatives of sterols are suitable surfactants for use in the present invention. These surfactants can be hydrophilic or lipophilic. A preferred sterol in this class is cholesterol or the esters of cholesterol with an organic acid, such cholesteryl succinate. Preferred derivatives include the polyethylene glycol derivatives. These derivatives could be esters and ethers depending upon the chemical bonds formed between the polyethylene glycol moiety and the sterol moiety. Preferred hydrophilic surfactants in this class include PEG-24 cholesterol ether (Solulan C-24) and cholesteryl polyethylene glycol succinate, containing various molecular weights of the polyethylene glycol moiety. Examples of surfactants of this class are shown in Table 10.

TABLE 10
Sterol and Sterol Derivative Surfactants

| | COMMERCIAL | |
|---|---|---|
| COMPOUND | PRODUCT (Supplier) | HLB |
| Cholesterol, sitosterol, lanosterol | | <10 |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-5 (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

2.11. Polyethylene Glycol Sorbitan Fatty Acid Esters

A variety of PEG-sorbitan fatty acid esters are available and are suitable for use as surfactants in the present invention. In general, these surfactants are hydrophilic, although several lipophilic surfactants of this class can be used. Among the PEG-sorbitan fatty acid esters, preferred hydrophilic surfactants include PEG-20 sorbitan monolaurate (Tween-20), PEG-sorbitan monopalmitate (Tween-40), PEG-20 sorbitan monostearate (Tween-60), and PEG-20 sorbitan monooleate (Tween-80). Examples of these surfactants are shown in Table 11.

TABLE 11
PEG-Sorbitan Fatty Acid Esters

| COMPOUND | COMMERCIAL PRODUCT (Supplier) | HLB |
|---|---|---|
| PEG-10 sorbitan laurate | Liposorb L-10 (Lipo Chem.) | >10 |
| PEG-20 sorbitan monolaurate | Tween-20 (Atlas/ICI), Crillet 1 (Croda), DACOL | 17 |
| | MLS 20 (Condea) | |
| PEG-4 sorbitan monolaurate | Tween-21 (Atlas/ICI), Crillet 11 (Croda) | 13 |
| PEG-80 sorbitan monolaurate | Hodag PSML-80 (Calgene); T-Maz 28 | >10 |
| PEG-6 sorbitan monolaurate | Nikkol GL-1 (Nikko) | 16 |
| PEG-20 sorbitan monopalmitate | Tween-40 (Atlas/ICI), Crillet 2 (Croda) | 16 |
| PEG-20 sorbitan monostearate | Tween-60 (Atlas/ICI), Crillet 3 (Croda) | 15 |
| PEG-4 sorbitan monostearate | Tween-61 (Atlas/ICI), Crillet 31 (Croda) | 9.6 |
| PEG-8 sorbitan monostearate | DACOL MSS (Condea) | >10 |
| PEG-6 sorbitan monostearate | Nikkol TS106 (Nikko) | 11 |
| PEG-20 sorbitan tristearate | Tween-65 (Atlas/ICI), Crillet 35 (Croda) | 11 |
| PEG-6 sorbitan tetrastearate | Nikkol GS-6 (Nikko) | 3 |
| PEG-60 sorbitan tetrastearate | Nikkol GS-460 (Nikko) | 13 |
| PEG-5 sorbitan monooleate | Tween-81 (Atlas/ICI), Crillet 41 (Croda) | 10 |
| PEG-6 sorbitan monooleate | Nikkol TO-106 (Nikko) | 10 |
| PEG-20 sorbitan monooleate | Tween-80 (Atlas/ICI), Crillet 4 (Croda) | 15 |
| PEG-40 sorbitan oleate | Emalex ET 8040 (Nihon Emulsion) | 18 |
| PEG-20 sorbitan trioleate | Tween-85 (Atlas/ICI), Crillet 45 (Croda) | 11 |
| PEG-6 sorbitan tetraoleate | Nikkol GO-4 (Nikko) | 8.5 |
| PEG-30 sorbitan tetraoleate | Nikkol GO-430 (Nikko) | 12 |
| PEG-40 sorbitan tetraoleate | Nikkol GO-440 (Nikko) | 13 |
| PEG-20 sorbitan | Tween-120 (Atlas/ICI), Crillet 6 (Croda) | >10 |
| monoisostearate | | |
| PEG sorbitol hexaoleate | Atlas G-1086 (ICI) | 10 |
| PEG-6 sorbitol hexastearate | Nikkol GS-6 (Nikko) | 3 |

2.12. Polyethylene Glycol Alkyl Ethers

Ethers of polyethylene glycol and alkyl alcohols are suitable surfactants for use in the present invention. Preferred lipophilic ethers include PEG-3 oleyl ether (Volpo® 3) and PEG-4 lauryl ether (Brij® 30). Examples of these surfactants are shown in Table 12.

TABLE 12
Polyethylene Glycol Alkyl Ethers

| | COMMERCIAL | |
|---|---|---|
| COMPOUND | PRODUCT (Supplier) | HLB |
| PEG-2 oleyl ether, oleth-2 | Brij 92/93 (Atlas/ICI) | 4.9 |
| PEG-3 oleyl ether, oleth-3 | Volpo 3 (Croda) | <10 |
| PEG-5 oleyl ether, oleth-5 | Volpo 5 (Croda) | <10 |
| PEG-10 oleyl ether, oleth-10 | Volpo 10 (Croda), Brij 96/97 | 12 |
| | (Atlas/ICI) | |
| PEG-20 oleyl ether, oleth-20 | Volpo 20 (Croda), Brij 98/99 | 15 |
| | (Atlas/ICI) | |
| PEG-4 lauryl ether, laureth-4 | Brij 30 (Atlas/ICI) | 9.7 |
| PEG-9 lauryl ether | | >10 |
| PEG-23 lauryl ether, laureth-23 | Brij 35 (Atlas/ICI) | 17 |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

2.13. Sugar Esters

Esters of sugars are suitable surfactants for use in the present invention. Preferred hydrophilic surfactants in this class include sucrose monopalmitate and sucrose monolaurate. Examples of such surfactants are shown in Table 13.

TABLE 13
Sugar Ester Surfactants

| COMPOUND | COMMERCIAL PRODUCT (Supplier) | HLB |
|---|---|---|
| Sucrose distearate | SUCRO ESTER 7 (Gattefosse), | 3 |
| | Crodesta F-10 (Croda) | |
| Sucrose | SUCRO ESTER 11 (Gattefosse), | 12 |
| distearate/monostearate | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate 1695 | 15 |
| | (Mitsubishi-Kasei) | |

2.14. Polyethylene Glycol Alkyl Phenols

Several hydrophilic PEG-alkyl phenol surfactants are available, and are suitable for use in the present invention. Examples of these surfactants are shown in Table 14.

TABLE 14
Polyethylene Glycol Alkyl Phenol Surfactants

| | COMMERCIAL | |
|---|---|---|
| COMPOUND | PRODUCT (Supplier) | HLB |
| PEG-10-100 nonyl phenol | Triton X series (Rohm & Haas), | >10 |
| | Igepal CA series (GAF, | |
| | U.S.A), Antarox CA series | |
| | (GAF, U.K) | |
| PEG-15-100 octyl phenol ether | Triton N-series (Rohm & Haas), | >10 |
| | Igepal CO series (GAF, | |
| | U.S.A), Antarox CO series | |
| | (GAF, U.K) | |

2.15. Polyoxyethylene-Polyoxypropylene Block Copolymers

The POE-POP block copolymers are a unique class of polymeric surfactants. The unique structure of the surfactants, with hydrophilic POE and lipophilic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. The generic term for these polymers is “poloxamer” (CAS 9003-11-6). These polymers have the formula:

HO(C₂H₄O)ₐ(C₃H₆O)_{b}(C₂H₄O)ₐH

where “a” and “b” denote the number of polyoxyethylene and polyoxypropylene units, respectively.

Preferred hydrophilic surfactants of this class include Poloxamers 108, 188, 217, 238, 288, 338, and 407. Preferred lipophilic surfactants in this class include Poloxamers 124, 182, 183, 212, 331, and 335.

Examples of suitable surfactants of this class are shown in Table 15. Since the compounds are widely available, commercial sources are not listed in the Table. The compounds are listed by generic name, with the corresponding “a” and “b” values.

TABLE 15
POE-POP Block Copolymers

| COMPOUND | a, b values in HO(C₂H₄O)ₐ(C₃H₆O)_{b}(C₂H₄O)ₐH | | HLB |
|---|---|---|---|
| Poloxamer 105 | a = 11 | b = 16 | 8 |
| Poloxamer 108 | a = 46 | b = 16 | >10 |
| Poloxamer 122 | a = 5 | b = 21 | 3 |
| Poloxamer 123 | a = 7 | b = 21 | 7 |
| Poloxamer 124 | a = 11 | b = 21 | >7 |
| Poloxamer 181 | a = 3 | b = 30 | |
| Poloxamer 182 | a = 8 | b = 30 | 2 |
| Poloxamer 183 | a = 10 | b = 30 | |
| Poloxamer 184 | a = 13 | b = 30 | |
| Poloxamer 185 | a = 19 | b = 30 | |
| Poloxamer 188 | a = 75 | b = 30 | 29 |
| Poloxamer 212 | a = 8 | b = 35 | |
| Poloxamer 215 | a = 24 | b = 35 | |
| Poloxamer 217 | a = 52 | b = 35 | |
| Poloxamer 231 | a = 16 | b = 39 | |
| Poloxamer 234 | a = 22 | b = 39 | |
| Poloxamer 235 | a = 27 | b = 39 | |
| Poloxamer 237 | a = 62 | b = 39 | 24 |
| Poloxamer 238 | a = 97 | b = 39 | |
| Poloxamer 282 | a = 10 | b = 47 | |
| Poloxamer 284 | a = 21 | b = 47 | |
| Poloxamer 288 | a = 122 | b = 47 | >10 |
| Poloxamer 331 | a = 7 | b = 54 | 0.5 |
| Poloxamer 333 | a = 20 | b = 54 | |
| Poloxamer 334 | a = 31 | b = 54 | |
| Poloxamer 335 | a = 38 | b = 54 | |
| Poloxamer 338 | a = 128 | b = 54 | |
| Poloxamer 401 | a = 6 | b = 67 | |
| Poloxamer 402 | a = 13 | b = 67 | |
| Poloxamer 403 | a = 21 | b = 67 | |
| Poloxamer 407 | a = 98 | b = 67 | |

Other block co-polymers are also suitable for the present invention. The block co-polymers can be made of various block components in different combination and sequences, such as BA diblock, ABA triblock, BAB triblock, and other more complex combinations and sequences involving three or more block components. The block components can be any poly(alkylene oxide), poly(lactic acid), poly(glycolic acid), poly(lactic-co-glycolic acid), poly(vinylpyrrolidone) and poly(ε-caprolactone). The molecular weights of suitable block co-polymers can range from a few thousand to a few million Daltons. These block co-polymers can be either hydrophilic or lipophilic depending on the distribution and ratios of different block components. Other co-polymers, not necessarily block co-polymers, are also suitable for the present invention. The co-polymers can be made of monomers of any combinations. The monomer component can be any alkylene oxide, lactic acid, glycolic acid, vinylpyrrolidone, or ε-caprolactone.

2.16. Sorbitan Fatty Acid Esters

Sorbitan esters of fatty acids are suitable surfactants for use in the present invention. Among these esters, preferred hydrophobic surfactants include sorbitan monolaurate (Arlacel® 20), sorbitan monopalmitate (Span-40®), sorbitan monooleate (Span-80®), sorbitan monostearate, and sorbitan tristearate. Examples of these surfactants are shown in Table 16.

TABLE 16
Sorbitan Fatty Acid Ester Surfactants

| COMPOUND | COMMERCIAL PRODUCT (Supplier) | HLB |
|---|---|---|
| Sorbitan monolaurate | Span-20 (Atlas/ICI), Crill 1 (Croda), | 8.6 |
| | Arlacel 20 (ICI) | |
| Sorbitan monopalmitate | Span-40 (Atlas/ICI), Crill 2 (Croda), | 6.7 |
| | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan monoisostearate | Crill 6 (Croda), Nikkol SI-10 (Nikko) | 4.7 |
| Sorbitan sesquistearate | Nikkol SS-15 (Nikko) | 4.2 |

2.17. Lower Alcohol Fatty Acid Esters

Esters of lower alcohols (C₂₋₄) and fatty acids (C₈₋₁₈) are suitable surfactants for use in the present invention. Among these esters, preferred lipophilic surfactants include ethyl oleate (Crodamol® EO), isopropyl myristate (Crodamol® IPM), and isopropyl palmitate (Crodamol® IPP). Examples of these surfactants are shown in Table 17.

TABLE 17
Lower Alcohol Fatty Acid Ester Surfactants

| COMPOUND | COMMERCIAL PRODUCT (Supplier) | HLB |
|---|---|---|
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

2.18. Ionic Surfactants

Ionic surfactants, including cationic, anionic and zwitterionic surfactants, are suitable hydrophilic surfactants for use in the present invention. Preferred anionic surfactants include fatty acid salts and bile salts. Preferred cationic surfactants include carnitines. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Examples of such surfactants are shown in Table 18. For simplicity, typical counterions are shown in the entries in the Table. It will be appreciated by one skilled in the art, however, that any bioacceptable counterion may be used. For example, although the fatty acids are shown as sodium salts, other cation counterions can also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in the Table.

TABLE 18
Ionic Surfactants

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glyco chenodeoxycholate | |
| Sodium cholylsarcosinate | |
| Sodium N-methyl taurocholate | |
| PHOSPHOLIPIDS | |
| Egg/Soy lecithin [Epikuron ® (Lucas Meyer), | |
| Ovothin® (Lucas Meyer)] | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol | |
| ethoxylates with phosphoric acid or anhydride | |
| CARBOXYLATES | |
| Ether carboxylates (by oxidation of terminal OH group of fatty | |
| alcohol ethoxylates) | |
| Succinylated monoglycerides [LAMEGIN ZE (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids | |
| calcium/sodium stearoyl-2-lactylate | |
| calcium/sodium stearoyl lactylate | |
| Alginate salts | |
| Propylene glycol alginate | |

SULFATES AND SULFONATES
Ethoxylated alkyl sulfates
Alkyl benzene sulfones
α-olefin sulfonates
Acyl isethionates
Acyl taurates
Alkyl glyceryl ether sulfonates
Octyl sulfosuccinate disodium
Disodium undecylenamideo-MEA-sulfosuccinate

| | |
|---|---|
| CATIONIC SURFACTANTS | >10 |
| Hexadecyl triammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine): | |
| Lauryl betaine (N-lauryl,N,N-dimethylglycine) | |
| Ethoxylated amines: | |
| Polyoxyethylene-15 coconut amine | |

2.19 Unionized Ionizable Surfactants

Ionizable surfactants, when present in their unionized (neutral, non-salt) form, are lipophilic surfactants suitable for use in the compositions of the present invention. Particular examples of such surfactants include free fatty acids, particularly C₆₋₂₂ fatty acids, and bile acids. More specifically, suitable unionized ionizable surfactants include the free fatty acid and bile acid forms of any of the fatty acid salts and bile salts shown in Table 18.

2.20 Derivatives of Fat-Soluble Vitamins

Derivatives of oil-soluble vitamins, such as vitamins A, D, E, K, etc., are also useful surfactants for the compositions of the present invention. An example of such a derivative is tocopheryl PEG-1000 succinate (TPGS, available from Eastman) and other tocopheryl PEG succinate derivatives with various molecular weights of the PEG moiety, such as PEG 100-8000.

2.21 Preferred Surfactants

Among the above-listed surfactants, several combinations are preferred. In some of the preferred combinations, the vehicle includes at least one hydrophilic surfactant. Preferred non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters; sugar ethers; sucroglycerides; and mixtures thereof.

More preferably, the non-ionic hydrophilic surfactant is selected from the group consisting of polyoxyethylene alkylethers; polyethylene glycol fatty acid esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. The glyceride can be a monoglyceride, diglyceride, triglyceride, or a mixture.

Also preferred are non-ionic hydrophilic surfactants that are reaction mixtures of polyols and fatty acids, glycerides, vegetable oils, hydrogenated vegetable oils, or sterols. These reaction mixtures are largely composed of the transesterification products of the reaction, along with commonly complex mixtures of other reaction products. The polyol is preferably glycerol, ethylene glycol, polyethylene glycol, sorbitol, propylene glycol, pentaerythritol, or a saccharide.

Several particularly preferred compositions are those that include as a non-ionic hydrophilic surfactant such as PEG-10 laurate, PEG-12 laurate, IIEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-1000 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, or a poloxamer.

Among these preferred surfactants, more preferred are PEG-20 laurate, PEG-20 oleate, PEG-35 castor oil, PEG-40 palm kernel oil, PEG-40 hydrogenated castor oil, PEG-60 corn oil, PEG-25 glyceryl trioleate, polyglyceryl-10 laurate, PEG-6 caprate/caprylate glycerides, PEG-8 caprate/caprylate glycerides, PEG-30 cholesterol, polysorbate 20 polysorbate 80, POE-9 lauryl ether, POE-23 lauryl ether, POE-10 oleyl ether, PEG-24 cholesterol, sucrose monostearate, sucrose monolaurate, and poloxamers. Most preferred are PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, and hydrophilic poloxamers.

The hydrophilic surfactant can also be, or can include as a component, an ionic surfactant. Preferred ionic surfactants include alkyl ammonium salts; bile acids and salts, analogues, and derivatives thereof; fusidic acid and derivatives thereof, fatty acid derivatives of amino acids, oligopeptides, and polypeptides; glyceride derivatives of amino acids oligopeptides, and polypeptides; acyl lactylates; mono-and di-acetylated tartaric acid esters of mono- and di-glycerides; succinylated monoglycerides; citric acid esters of mono- and di-glycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

More preferable ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono- and di-acetylated tartaril acid esters of mono- and di-glycerides; succinylated monoglycerides; citric acid esters of mono- and di-glycerides; carnitines; and mixtures thereof.

More specifically, preferred ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono- and di-acetylated tartaric acid esters of mono- and di-glycerides, citric acid esters of mono- and di-glycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof

Particularly preferred ionic surfactants are lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono- and di-acetylated tartaric acid esters of mono- and di-glycerides, citric acid esters of mono- and di-glycerides cholate, taurocholate glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono- and di-acetylated tartaric acid esters of mono- and di-glycerides, citric acid esters of mono- and di-glycerides, taurocholate, caprylate, caprate, oleate, lauryl sulfate, docusate, and salts and mixtures thereof.

Preferred lipophilic surfactants are alcohols; polyoxyethylene alkylethers; fatty acids; glycerol fatty acid esters; acetylated glycerol fatty acid esters; lower alcohol fatty acid esters; polyethylene glycol fatty acid esters; polyethylene glycol glycerol fatty acid esters; polypropylene glycol fatty acid esters; polyoxyethylene glycerides; lactic acid derivatives of mono- and di-glycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils.

As with the hydrophilic surfactants, lipophilic surfactants can be reaction mixtures of polyols and fatty acids, glycerides, vegetable oils, hydrogenated vegetable oils, and sterols.

Preferably, the lipophilic surfactant is selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono- and di-glycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols.

More preferred are lower alcohol fatty acids esters; polypropylene glycol fatty acid esters; propylene glycol fatty acid esters; glycerol fatty acid esters; acetylated glycerol fatty acid esters; lactic acid derivatives of mono- and di-glycerides; sorbitan fatty acid esters; polyoxyethylene vegetable oils; and mixtures thereof, with glycerol fatty acid esters and acetylated glycerol fatty acid esters being most preferred. Among the glycerol fatty acid esters, the esters are preferably mono- or diglycerides, or mixtures of mono- and diglycerides, where the fatty acid moiety is a C₆₋₂₂ fatty acid.

Also preferred are lipophilic surfactants that are the reaction mixture of polyols and fatty acids, glycerides, vegetable oils, hydrogenated vegetable oils, and sterols. Preferred polyols are polyethylene glycol, sorbitol, propylene glycol, and pentaerythritol.

Specifically preferred lipophilic surfactants include myristic acid; oleic acid; lauric acid; stearic acid; palmitic acid; PEG 1-4 stearate; PEG 2-4 oleate; PEG-4 dilaurate; PEG-4 dioleate; PEG-4 distearate; PEG-6 dioleate; PEG-6 distearate; PEG-8 dioleate; PEG 3-16 castor oil; PEG 5-10 hydrogenated castor oil; PEG 6-20 corn oil; PEG 6-20 almond oil; PEG-6 olive oil; PEG-6 peanut oil; PEG-6 palm kernel oil; PEG-6 hydrogenated palm kernel oil; PEG-4 capric/caprylic triglyceride, mono, di, tri, tetra esters of vegetable oil and sorbitol; pentaerythrityl di or tetra stearate, isostearate, oleate, caprylate, or caprate; polyglyceryl 2-4 oleate, stearate, or isostearate; polyglyceryl 4-10 pentaoleate; polyglyceryl-3 dioleate; polyglyceryl-6 dioleate; polyglyceryl-1 0 trioleate; polyglyceryl-3 distearate; propylene glycol mono- or diesters of a C₆₋₂₀ fatty acid; monoglycerides of C₆₋₂₀ fatty acids; acetylated monoglycerides of C₆₋₂₀ fatty acids; diglycerides of C₆₋₂₀ fatty acids; tocopheryl PEG-800 succinate; lactic acid derivatives of monoglycerides; lactic acid derivatives of diglycerides; cholesterol; phytosterol; PEG 5-20 soya sterol; PEG-6 sorbitan tetra, hexastearate; PEG-6 sorbitan tetraoleate; sorbitan monolaurate; sorbitan monopalmitate; sorbitan mono, trioleate; sorbitan mono, tristearate; sorbitan monoisostearate; sorbitan sesquioleate; sorbitan sesquistearate; PEG 2-5 oleyl ether; POE 2-4 lauryl ether; PEG-2 cetyl ether; PEG-2 stearyl ether; sucrose distearate; sucrose dipalmitate; ethyl oleate; isopropyl myristate; isopropyl palmitate; ethyl linoleate; isopropyl linoleate; and poloxamers.

Among the specifically preferred lipophilic surfactants, most preferred are oleic acid; lauric acid; glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; tocopheryl PEG-800 succinate; and poloxamers.

3. Triglycerides

For compositions of the present invention that include a lipophilic additive, the lipophilic component can be a lipophilic surfactant or a triglyceride. Preferred triglycerides are those which solidify at ambient room temperature, with or without addition of appropriate additives, or those which in combination with particular surfactants and/or active ingredients solidify at room temperature. Examples of triglycerides suitable for use in the present invention are shown in Table 19. In general, these triglycerides are readily available from commercial sources. For several triglycerides, representative commercial products and/or commercial suppliers are listed.

TABLE 19
Triglycerides

| TRIGLYCERIDE | COMMERCIAL SOURCE |
|---|---|
| Aceituno oil | |
| Almond oil | Super Refined Almond Oil |
| | (Croda) |
| Arachis oil | |
| Babassu oil | |
| Blackcurrant seed oil | |
| Borage oil | |
| Buffalo ground oil | |
| Candlenut oil | |
| Canola oil | Lipex 108 (Abitec) |
| Caster oil | |
| Chinese vegetable tallow oil | |
| Cocoa buffer | |
| Coconut oil | |
| Coffee seed oil | Pureco 76 (Abitec) |
| Corn oil | Super Refined Corn Oil |
| | (Croda) |
| Cottonseed oil | Super Refined Cottonseed Oil |
| | (Croda) |
| Crambe oil | |
| Cuphea species oil | |
| Evening primrose oil | |
| Grapeseed oil | |
| Groundnut oil | |
| Hemp seed oil | |
| Illipe butter | |
| Kapok seed oil | |
| Linseed oil | |
| Menhaden oil | Super Refined Menhaden Oil |
| | (Croda) |
| Mowrah butter | |
| Mustard seed oil | |
| Oiticica oil | |
| Olive oil | Super Refined Olive Oil |
| | (Croda) |
| Palm oil | |
| Palm kernel oil | |
| Peanut oil | Super Refined Peanut Oil |
| | (Croda) |
| Poppy seed oil | |
| Rapeseed oil | |
| Rice bran oil | |
| Safflower oil | Super Refined Safflower |
| | Oil (Croda) |
| Sal fat | |
| Sesame oil | Super Refined Sesame Oil |
| | (Croda) |
| Shark liver oil | Super Refined Shark Liver Oil |
| | (Croda) |
| Shea nut oil | |
| Soybean oil | Super Refined Soybean Oil |
| | (Croda) |
| Stillingia oil | |
| Sunflower oil | |
| Tall oil | |
| Tea sead oil | |
| Tobacco seed oil | |
| Tung oil (China wood oil) | |
| Ucuhuba | |
| Vernonia oil | |
| Wheat germ oil | Super Refined Wheat Germ Oil |
| | (Croda) |
| Hydrogenated caster oil | Castorwax |
| Hydrogenated coconut oil | Pureco 100 (Abitec) |
| Hydrogenated cottonseed oil | Dritex C (Abitec) |
| Hydrogenated palm oil | Dritex PST (Abitec); |
| | Softisan154 (Huls) |
| Hydrogenated soybean oil | Sterotex HM NF (Abitec); |
| | Dritex S (Abitec) |
| Hydrogenated vegetable oil | Sterotex NF (Abitec): Hydrokote |
| | M (Abitec) |
| Hydrogenated cottonseed/castor oil | Sterotex K (Abitec) |
| Partially hydrogenated soybean oil | Hydrokote AP5 (Abitec) |
| Partially soy and cottonseed oil | Apex B (Abitec) |
| Glyceryl tributyrate | (Sigma) |
| Glyceryl tricaproate | (Sigma) |
| Glyceryl tricaprylate | (Sigma) |
| Glyceryl tricaprate | Captex 1000 (Abitec) |
| Glyceryl trundecanoate | Captex 8227 (Abitec) |
| Glyceryl trilaurate | (Sigma) |
| Glyceryl trimyristate | Dynasan 114 (Huls) |
| Glyceryl tripalmitate | Dynasan 116 (Huls) |
| Glyceryl tristearate | Dynasan 118 (Huls) |
| Glyceryl triarcidate | (Sigma) |
| Glyceryl trimyristoleate | (Sigma) |
| Glyceryl tripalmitoleate | (Sigma) |
| Glyceryl trioleate | (Sigma) |
| Glyceryl trilinoleate | (Sigma) |
| Glyceryl trilinolenate | (Sigma) |
| Glyceryl tricaprylate/caprate | Captex 300 (Abitec); |
| | Captex 355 (Abitec); |
| | Miglyol 810 (Huls); |
| | Miglyol 812 (Huls) |
| Glyceryl tricaprylate/caprate/laurate | Captex 350 (Abitec) |
| Glyceryl tricaprylate/caprate/linoleate | Captex 810 (Abitec); |
| | Miglyol 818 (Huls) |
| Glyceryl tricaprylate/caprate/stearate | Softisan 378 (Huls); (Larodan) |
| Glyceryl tricaprylate/laurate/stearate | (Larodan) |
| Glyceryl 1,2-caprylate-3-linoleate | (Larodan) |
| Glyceryl 1,2-caprate-3-stearate | (Larodan) |
| Glyceryl 1,2-laurate-3-myristate | (Larodan) |
| Glyceryl 1,2-myristate-3-laurate | (Larodan) |
| Glyceryl 1,3-palmitate-2-butyrate | (Larodan) |
| Glyceryl 1,3-stearate-2-caprate | (Larodan) |
| Glyceryl 1,2-linoleate-3-caprylate | (Larodan) |

Fractionated triglycerides, modified triglycerides, synthetic triglycerides, and mixtures of triglycerides are also within the scope of the invention.

Preferred triglycerides include vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants” , may be suitable to provide all or part of the triglyceride component for the compositions of the present invention. Examples of commercial surfactant compositions containing triglycerides include some members of the surfactant families Gelucires (Gattefosse), Maisines (Gattefosse), and Imwitors (Hüls). Specific examples of these compositions are: Gelucire 44/14 (saturated polyglycolized glycerides); Gelucire 50/13 (saturated polyglycolized glycerides); Gelucire 53/10 (saturated polyglycolized glycerides); Gelucire 33/01 (semi-synthetic triglycerides of C₈-C₁₈ saturated fatty acids); Gelucire 39/01 (semi-synthetic glycerides); other Gelucires, such as 37/06, 43/01, 35/10, 37/02, 46/07, 48/09, 50/02, 62/05, etc.; Maisine 35-I (linoleic glycerides); and Imwitor 742 (caprylic/capric glycerides).

Still other commercial surfactant compositions having significant triglyceride content are known to those skilled in the art. It should be appreciated that such compositions, which contain triglycerides as well as surfactants, may be suitable to provide all or part of the triglyceride component of the compositions of the present invention, as well as all or part of the surfactant component.

4. Solubilizers

Various embodiments of the invention include one or more solubilizers, which can serve as an additive to increase the solubility of the pharmaceutical active ingredient or other composition components in the solid carrier. In addition, the solubilizer can be used to facilitate the dissolution/solubilization of the active ingredient from the dosage form in an aqueous medium, such as intestinal fluids or simulated intestinal fluids. Suitable solubilizers for use in the compositions of the present invention include:

alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives;

ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide);

amides, such as 2-pyrrolidone, 2-piperidone, s-caprolactam, N-alkylpyrrolidone, N-hydroxyalkylpyrrolidone, N-alkylpiperidone, N-alkylcaprolactam, dimethylacetamide, and polyvinylpyrrolidone;

esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and

and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water.

Mixtures of solubilizers are also within the scope of the invention. Except as indicated, these compounds are readily available from standard commercial sources.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropylmethylcellulose, hydroxypropyl cyclodextrins, ethanol, polyethylene glycol 200-600, glycofurol, transcutol, propylene glycol, and dimethyl isosorbide. Particularly preferred solubilizers include sorbitol, polyvinylpyrrolidone, hydroxypropyl methylcellulose, hydroxypropyl cyclodextrins, glycerol, triacetin, ethyl alcohol, polyethylene glycol, glycofurol and propylene glycol. Most preferred solubilizers include polyvinylpyrrolidone, hydroxypropylmethylcellulose, hydroxypropyl cyclodextrins, polyethylene glycol, and combinations thereof.

The amount of solubilizer that can be included in compositions of the present invention is not particularly limited. Of course, when such compositions are ultimately administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of active ingredient, with excess solubilizer removed prior to providing the composition to a patient using conventional techniques, such as distillation or evaporation.

5. Substrates

The substrate of the compositions of the present invention can be a powder or a multiparticulate, such as a granule, a pellet, a bead, a spherule, a beadlet, a microcapsule, a millisphere, a nanocapsule, a nano sphere, a micro sphere, a platelet, a minitablet, a tablet or a capsule. A powder constitutes a finely divided (milled, micronized, nanosized, precipitated) form of an active ingredient or additive molecular aggregates or a compound aggregate of multiple components or a physical mixture of aggregates of an active ingredient and/or additives. Such substrates can be formed of various materials known in the art, such as, for example: sugars, such as lactose, sucrose or dextrose; polysaccharides, such as maltodextrin or dextrates;

starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin.

The substrate can also be formed of any of the active ingredients, surfactants, triglycerides, solubilizers or additives described herein. In one particular embodiment, the substrate is a solid form of an additive, an active ingredient, and a surfactant and/or a triglyceride and/or a solubilizer; a complex of an additive, and a surfactant, triglyceride, and/or solubilizer, and an active ingredient; a coprecipitate of an additive, a surfactant, triglyceride and/or a solubilizer, and an active ingredient, or a mixture thereof.

It should be emphasized that the substrate need not be a solid material, although often it will be a solid. For example, the encapsulation coat on the substrate may act as a solid “shell” surrounding and encapsulating a liquid or semi-liquid substrate material. Such substrates are also within the scope of the present invention, as it is ultimately the carrier, of which the substrate is a part, which must be a solid.

6. Additives

The solid pharmaceutical compositions of the present invention can optionally include one or more additives, sometimes referred to as excipients. The additives can be contained in an encapsulation coat in compositions which include an encapsulation coat, or can be part of the solid carrier, such as coated to an encapsulation coat, or contained within the components forming the solid carrier. Alternatively, the additives can be contained in the pharmaceutical composition but not part of the solid carrier itself. Specific, non-limiting examples of additives are described below.

Suitable additives are those commonly utilized to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The additive can also be pre-coated or encapsulated. Appropriate coatings are well known in the art, and are further described in the sections below. Based on the functionality of the additives, examples of the additives are as follows:

Exemplary additives that are conventionally used in pharmaceutical compositions can be included, and these additives are well known in the art. Such additives include:

anti-adherents (anti-sticking agents, glidants, flow promoters, lubricants) such as talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate;

anticoagulants, such as acetylated monoglycerides;

antifoaming agents, such as long-chain alcohols and silicone derivatives;

antioxidants, such as BHT, BHA, gallic acid, propyl gallate, ascorbic acid, ascorbyl palmitate, 4-hydroxymethyl-2,6-di-tert-butyl phenol, and tocopherol;

binders (adhesives), i.e., agents that impart cohesive properties to powdered materials through particle-particle bonding, such as matrix binders (dry starch, dry sugars), film binders (PVP, starch paste, celluloses, bentonite, sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose). Preferred binders include water soluble polymers such as polyvinyl alcohol, polyvinylpyrrolidone, methylcellulose, hydroxypropyl cellulose, hydroxymethyl cellulose and the like. A water soluble binder is preferably applied from an aqueous medium such as water at a level of about 0.1-5 wt %, and preferably about 0.25-3 wt % of binder based on the total weight of the formulation;

bufferants, where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, tris-ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid;

chelating agents, such as EDTA and EDTA salts;

coagulants, such as alginates;

colorants or opaquants, such as titanium dioxide, food dyes, lakes, natural vegetable colorants, iron oxides, silicates, sulfates, magnesium hydroxide and aluminum hydroxide;

coolants, such as halogenated hydrocarbons (e.g., trichloroethane, trichloroethylene, dichloromethane, fluorotrichloromethane), diethylether and liquid nitrogen;

cryoprotectants, such as trehelose, phosphates, citric acid, tartaric acid, gelatin, dextran and mannitol;

diluents or fillers, such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose. Preferred fillers include, polyethylene glycol (molecular weight 600-30,000 and preferably 3,350-8,000) and sugars such as lactose, dextrose, sucrose, maltose, microcrystalline cellulose and the like. When included, the filler may comprise about 5-95 wt %, and preferably about 20-80 wt % based on the total weight of the formulation; disintegrants or super disintegrants, such as croscarmellose sodium, starch, starch derivatives, clays, gums, cellulose, cellulose derivatives, alginates, crosslinked polyvinypyrrolidone, sodium starch glycolate and microcrystalline cellulose;

hydrogen bonding agents, such as magnesium oxide;

flavorants or desensitizers, such as spray-dried flavors, essential oils and ethyl vanillin;

ion-exchange resins, such as styrene/divinyl benzene copolymers, and quaternary ammonium compounds;

plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate;

preservatives, such as ascorbic acid, boric acid, sorbic acid, benzoic acid, and salts thereof, parabens, phenols, benzyl alcohol, and quaternary ammonium compounds;

solubilizers, as described herein;

solvents, such as alcohols, ketones, esters, chlorinated hydrocarbons and water;

stabilization aids such as an alkaline material selected from the group consisting of basic inorganic salts, organic bases, and mixtures thereof. Suitable basic inorganic salts include the sodium, potassium, calcium, magnesium and aluminum salts of phosphoric acid, carbonic acid, and the hydroxides and oxides of sodium, potassium, calcium, magnesium and aluminum as well as aluminum/magnesium compounds. Suitable organic bases include triethanolamine, tris, triethylamine, basic amino acids and derivatives thereof, such as lysine and arginine, the sodium, potassium, calcium, magnesium and aluminum salts of an organic acid such as citric acid, and mixtures thereof. When included, the stabilization aid may be present at a level of about 5-500 wt %, and preferably about 10-100 wt % of the amount of the active ingredient, depending on the relative strength of the alkaline material. In addition, when a hydrophilic surfactant or lipophilic additive is included in the formulation, the amount of stabilization aid required for the same effectiveness of active ingredient stabilization may be reduced, or a previously ineffective stabilization aid (weaker base) can now be used effectively;

sweeteners, including natural sweeteners such as maltose, sucrose, glucose, sorbitol, glycerin and dextrins, and artificial sweeteners, such as aspartame, saccharine and saccharine salts; and

thickeners (viscosity modifiers, thickening agents), such as sugars, polyvinylpyrrolidone, cellulosics, polymers and alginates.

Additives can also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnuaba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose anti acetate trimellitate, carboxymethylethyl cellulose, hydroxypropylmethyl cellulose phthalate); inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives can be readily determined by one skilled in the art, according to the particular properties desired.

Preferred additives include bufferants such as pharmaceutically acceptable bases, salts of pharmaceutically acceptable cations, and combinations thereof, examples of which are set forth above. In a preferred embodiment, the bufferant is fast dissolving upon contact with moisture.

When the active ingredient is subject to enzymatic degradation, the compositions can include an enzyme inhibiting agent. Enzyme inhibiting agents are shown for example, in Bernskop-Schnurch, A., “The use of inhibitory agents to overcome enzymatic barrier to perorally administered therapeutic peptides and proteins”, J. Controlled Release 52, 1-16 (1998), the disclosure of which is incorporated herein by reference.

Generally, inhibitory agents can be divided into the following classes:

inhibitors that are not based on amino acids, such as P-aminobenzamidine, FK-448, camostat mesylate, sodium glycocholate;

amino acids and modified amino acids, such as aminoboronic acid derivatives and n-acetylcysteine;

peptides and modified peptides, such as bacitracin, phosphinic acid dipeptide derivatives, pepstatin, antipain, leupeptin, chymostatin, elastatin, bestatin, phosphoramindon, puromycin, cytochalasin potatocarboxy peptidase inhibitor, and amastatin;

polypeptide protease inhibitors, such as aprotinin (bovine pancreatic trypsin inhibitor), Bowman-Birk inhibitor and soybean trypsin inhibitor, chicken egg white trypsin inhibitor, chicken ovoinhibitor, and human pancreatic trypsin inhibitor. Complexing agents, such as EDTA, EGTA, 1,10-phenanthroline and hydroxychinoline; and

mucoadhesive polymers and polymer-inhibitor conjugates, such as polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bwoman-Birk inhibitor.

The choice and levels of the enzyme inhibitor are based on toxicity, specificity of the proteases and the potency of the inhibition. The inhibitor can be suspended or solubilized in the composition preconcentrate, or added to the aqueous diluent or as a beverage.

Without wishing to be bound by theory, it is believed that an inhibitor can function solely or in combination as: a competitive inhibitor, by binding at the substrate binding site of the enzyme, thereby preventing the access to the substrate; examples of inhibitors believed to operate by this mechanism are antipain, elastatinal and the Bowman Birk inhibitor; a non-competitive inhibitor which can be simultaneously bound to the enzyme site along with the substrate, as their binding sites are not identical; and/or a complexing agent due to loss in enzymatic activity caused by deprivation of essential metal ions out of the enzyme structure.

7. Dosage Forms

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery, although oral delivery is presently preferred.

The pharmaceutical composition and/or the solid carrier particles can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be effected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided:

Seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. In particular, the seal coating provides insulation from moisture and protection from chemical degradation, for example, acid-catalyzed degradation. The seal coating can also serve as a pH-buffering region, where proton or other acidic species diffuse from the outside in towards the core of the drug-containing carrier. The seal coating may further include a pharmaceutically acceptable base, a salt of a pharmaceutically acceptable cation or combinations thereof, that serve to neutralize the acid diffusing inwards, or to alkalinize the moisture diffusing in towards the drug-containing core. The buffering capacity or the alkalinizing effect is proportional to the amount and the basicity of the base or basic salt employed in the seal coating.

The isolating effect of the seal coating is proportional to the thickness of the coating. At least one layer of the seal coating can be directly applied to the outer surface of the solid carrier. If an enteric coating is present, the seal coating can be applied between the solid carrier and the enteric coating, or on the outer surface of the enteric coating, or both.

The materials suitable for use as the seal coating cane be selected from any pharmaceutically acceptable inert material, including polymers that are designed for film-coating applications. Exemplary seal coating materials include sugars, polyethylene glycol, polyvinylpyrrolidone, polyvinyl alcohol, hydroxypropylcellulose, methylcellulose, hydroxymethylcellulose, hydroxypropylmethylcellulose, polyvinyl acetal diethylaminoacetate, and the like.

Further exemplary seal coat materials include lipophilic surfactants, triglycerides, waxes, polymers, and combinations thereof. In a preferred embodiment, the seal coat is comprised of a digestible material, such as glycol monostearate, mono-, di- and triglycerides, polyethylene glycol stearate, distilled acetylated monoglyceride, polyglycolized glyceride, and so forth. Water soluble cellulose ethers are preferred for this application. HPMC and ethyl cellulose in combination, or Eudragit E100, may be particularly suitable for taste masking applications. Traditional enteric coating materials listed elsewhere can also be applied to form an isolating layer. The seal coating may further comprise appropriate additives, such as plasticizers, disintegrants, dissolution aids, and colorants. It may also comprise a stabilizing agent such as a bufferant. When a basic bufferant is included, an additional seal coat of a neutral material may be included to separate the basic bufferant from the outer enteric coating.

Extended release coating: The term “extended release coating” as used herein means a coating designed to effect delivery over an extended period of time. Preferably, the extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coating: The term “enteric coating” as used herein relates to a mixture of pharmaceutically acceptable excipients which is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. In some embodiments, such as when a moisture barrier is desired, the enteric coating may be included. However, as the enteric coating may increase the amount of degradation of the active ingredient, some embodiments of the invention may forego the enteric coating. The enteric coating may include an acid-resistant material, preferably one that can resists acids up to a pH of above about 5.0 or higher. Exemplary acid-resistant materials include, cellulose acetate phthalate, hydroxypropylmethylcellulose phthalate, polyvinyl acetate phthalate, carboxymethylethylcellulose, Eudragit L [1:1 ratio of poly(methacrylic acid):methylmethacrylate, Avg. MW 135,000, USP Type A)] or Eudragit S [1:2 ration of poly(methacrylic acid):methylmethacrylate, Avg. MW 135,000, USP Type B), and mixtures thereof. The enteric coating agent may also include an inert processing aid in an amount of about 10-80 wt %, and preferably about 30-50 wt % based on the total weight of the acid-resistant material and the inert processing aid. Exemplary materials suitable for uses as the inert processing aid includes, finely divided forms of talc, silicon dioxide, magnesium stearate etc. The enteric coating may further comprise a moisture-resistant component. Typical solvents which may be used to apply the acid resisting component-inert processing aid mixture include isopropyl alcohol, acetone, methylene chloride and the like. An aqueous suspension of the enteric coating agent can also be used for processing. Generally the acid-resistant material-inert processing aid mixture will comprise about 5-20 wt % of the mixture based on the total weight of the solvent and the mixture. Finally, when an enteric coat is included in the formulation, there may also be at least one layer of seal coating or separation coating between the drug-containing composition and the enteric coat. Such layers are typically made of an inert material such as an acid- and alkaline-resistant material. Additional additives and their levels, and selection of a primary coating material or materials will depend on the following properties:

resistance to dissolution and disintegration in the stomach;

impermeability to gastric fluids and drug/carrier/enzyme while in the stomach;

ability to dissolve or disintegrate rapidly at the target intestine site;

physical and chemical stability during storage;

non-toxicity;

easy application as a coating (substrate friendly); and

economical practicality.

Dosage forms of the compositions of the present invention can also be formulated as enteric coated delayed release oral dosage forms, i.e., as an oral dosage form of a pharmaceutical composition as described herein which utilizes an enteric coating to effect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. The preferred polymers for use in the present invention are anionic carboxylic polymers. The more preferred polymers and compatible mixtures thereof, and some of their properties, include, but are not limited to:

Shellac, also called purified lac, a refined product obtained from the resinous secretion of an insect. This coating dissolves in media of pH>7.

Acrylic polymers (preferred). The performance of acrylic polymers (primarily their solubility in biological fluids) can vary based on the degree and type of substitution. Examples of suitable acrylic polymers include methacrylic acid copolymers and ammonio methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for extended release. The Eudragit series E dissolve in the stomach. The Eudragit series L, L-30D and S are insoluble in stomach and dissolve in the intestine.

Cellulose Derivatives (also preferred). Examples of suitable cellulose derivatives are:

ethyl cellulose;

reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides;

cellulose acetate trimellitate (Eastman);

methylcellulose (Pharmacoat, Methocel);

hydroxypropyl methyl cellulose phthalate (HPMCP). The performance can vary based on the degree and type of substitution. HP-50, HP-55, HP-55S, HP-55F grades are suitable;

hydroxypropyl methyl cellulose succinate (HPMCS; AQOAT (Shin Etsu)). The performance can vary based on the degree and type of substitution. Suitable grades include AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH>5, and it is much less permeable to water vapor and gastric fluids; and

Cotteric (by Colorcon).

Combinations of the above materials can also be used.

The coating can, and usually does, contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxypropylcellulose, acid/base may be added to the coatings besides plasticizers to solubilize or disperse the coating material, and to improve coating performance and the coated product.

A particularly suitable methacrylic copolymer is Eudragit L®, particularly L-30D® and Eudragit 100-55®, manufactured by Rohm Pharma, Germany. In Eudragit L-30 D®, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract.

Another methacrylic acid polymer which is suitable for use in coating the composition or solid carrier which can be employed in the compositions and methods described herein, either alone or in combination with other coatings, is Eudragit S®, manufactured by Rohm Pharma, Germany. Eudragit S differs from Eudragit L-30-D only insofar as the ratio of free carboxyl groups to ester groups is approximately 1:2. Eudragit S is insoluble at pH below 5.5, but unlike Eudragit L-30-D, is poorly soluble in gastrointestinal fluids having pH of 5.5-7.0, such as is present in the small intestine media. This copolymer is soluble at pH 7.0 and above, i.e., the pH generally found in the colon. Eudragit S can be used alone as a coating to provide delivery of beginning at the large intestine via a delayed release mechanism. In addition, Eudragit S, being poorly soluble in intestinal fluids below pH 7, can be used in combination with Eudragit L-30-D, soluble in intestinal fluids above pH 5.5, in order to effect a delayed release composition. The more Eudragit L-30 D used, the more proximal release and delivery begins, and the more Eudragit S used, the more distal release and delivery begins. Both Eudragit L-30-D and Eudragit S can be substituted with other pharmaceutically acceptable polymers with similar pH solubility characteristics.

Preferred materials include shellac, acrylic polymers, cellulosic derivatives, polyvinyl acetate phthalate, and mixtures thereof. More preferred materials include Eudragit series E, L, S, RL, RS, NE, L®, L300®, S®, 100-55®, cellulose acetate phthalate, Aquateric, cellulose acetate trimellitate, ethyl cellulose, hydroxypropyl methyl cellulose phthalate, hydroxypropyl methyl cellulose succinate, poly vinyl acetate phthalate, and Cotteric. Most preferred materials include Eudragit series L, L300, S, L100-55, cellulose acetate phthalate, Aquateric, ethyl cellulose, hydroxypropyl methyl cellulose phthalate, hydroxypropyl methyl cellulose succinate, poly vinyl acetate phthalate, and Cotteric.

Extended release and targeted delayed release coatings for dosage forms of the compositions of the present invention are described more completely in Dansereau, et al., U.S. Pat. No. 5,622,721 and Kelm, et al., U.S. Pat. No. 5,686,105, the disclosures of which are incorporated herein by reference in their entirety.

Fast-Disintegrating Coatings for Immediate Release: Immediate release coating of solid carriers is commonly used to improve product elegance as well as for a moisture barrier, and taste and odor masking. Rapid breakdown of the film in gastric media is important, leading to effective disintegration and dissolution. Eudragit RD100 (Rohm) is an example of such a coating. It is a combination of a water insoluble cationic methacrylate copolymer with a water soluble cellulose ether. In powder form, it is readily dispensable into an easily sprayable suspension that dries to leave a smooth film. Such films rapidly disintegrate in aqueous media at a rate that is independent of pH and film thickness.

8. Processes

The compositions of the present invention can be prepared by a variety of processes to apply an encapsulation coat onto a substrate or to form a substrate-free solid carrier such as a multiparticulate or a powder. The commonly utilized coating and pelletization processes include balling, spheronization, extrusion, spray congealing, spray drying, pan coating, fluidized bed coating, melt extrusion, crystallization, cryopelletization, nanoencapsulation, coacervation, etc. It is also clear to one skilled in the art that appropriate additives can also be introduced to the composition or during the processes to facilitate the preparation of the solid carrier or the dosage forms, depending on the need of the individual process.

Coating processes typically involve spraying a coating solution onto a substrate. The coating solution can be a molten solution of the encapsulation coat composition free of a dispersing medium. The coating solution can also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level utilizing appropriate drying processes, such as vacuum evaporation, heating, freeze drying, etc.

Pelletization typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes can be adopted to control the level of the residual dispersing medium, if necessary.

A preferred drug formulation is based on pellets. The pellets may be prepared by processes that are well known to the art, such as spray congealing, spray drying, spin disc technique, extrusion, as described below. However, the formulation can also be in other forms, such as a tablet. The pellets with an inert core may be prepared by spray coating, granulation or other appropriate processes. An inert core or substrate may comprise a starch or sugar sphere such as nonpareil sugar seeds (having an average size of from 12 40 mesh, preferably about 16 to 25 mesh). The inert core is coated with at least one layer comprising the active ingredient. The core forming inert components are employed at 1:10 to 10:1 and preferably from 1:3 to 3:1 weight ratio to the active ingredient-containing layer(s). The active ingredient may comprise about 1-30 wt % and preferably about 5-20 wt % of the overall pellet composition. When the active ingredient is present in pellets with an inert core, the active ingredient may comprise about 20-70 wt %, and preferably about 40-50 wt % of the coat composition.

In one embodiment of the invention, the surface properties of the active agent particles are modified by a treatment with an interfacial modifying agent, for example by coating the particles with the interfacial modifying agent. This serves to minimize contact with moisture and other liquids (e.g., acids and bases). Suitable interfacial modifying agents include surfactants, polymers (preferably hydrophilic), lipids, gelatins, saccharides, and combinations thereof. The interfacial modifying agent may also be admixed with the particles (by, for example, co-grinding or co-micronizing), applied to the particles as a dry powder, and/or selected to chemically bind to the particle surface. If faster dissolution is desirable, the surface coating may be one that speeds wetting and comprises, for example, a hydrophilic surfactant, a sugar such as lactose, sucrose, or dextrose, or a hydrophilic polymer. If controlled release is desirable, e.g., sustained and/or delayed release, a suitable sustained and/or delayed release coating can be applied. Various pharmaceutically acceptable materials can be used for such coatings, including cellulose derivatives, polysaccharides, acrylic polymers, lipophilic surfactants, triglycerides, and mixtures thereof. Some of these materials can erode or dissolve slowly, whereas some do not dissolve in the acidic conditions of the stomach but will dissolve in the non-acidic conditions of the intestines. To further modify the release profile of the active agent from the solid particles, any of a variety of different particle preparation techniques may also be employed.

In another embodiment of the invention, the active agent is in the form of a plurality of solid particles, which may associate to form one or more larger dosage units such as a granule, pellet, bead or tablet, suspended in the vehicle. The particles may be single phase, or comprised of two or more phases. When the solid particles are wholly comprised of the active agent, the particulate phase can be amorphous or in a high energy state, i.e., a metastable crystalline phase or a stable crystalline phase (wherein the crystalline state may include any of various polymorphs or solvates), or it may be a mixture of at least one amorphous phase and at least one crystalline phase. For example, in a preferred embodiment, the active ingredient is present in its crystalline form. This is of particular interest for compounds such as lansoprazole, whose crystalline form is not readily soluble in water.

When the particles include one or more excipients, additives, or the like, the particulate material can be amorphous, crystalline, in the form of a solid solution with the excipient(s) and/or additive(s), or a mixture of two or more of these phases. The solid particles may contain a core comprised of the active agent, an excipient, or mixtures thereof, and may optionally be further coated with at least one layer of the active agent, the excipient, or mixtures thereof. The solid particles may be core-free, in the form of a powder or a plurality of granules, pellets, and/or beads, or combinations thereof.

The processes described above, the combination of the processes, or the modification of the processes are well know in the art. Some of the processes are briefly described herein for reference. Preferred methods include spray congealing, spray drying, and extrusion processes.

8.1. Balling, Spheronization or Extrusion

In a broad sense, pellets are very much like granules and bead; the techniques for producing pellets can also produce granules, beads, etc. Pellets, granules or beads are formed with the aid of a pelletizer, spheronizer or extruder. The pelletizer, spheronizer or extruder is able to form approximately spherical bodies from a mass of finely divided particles continuously, by a rolling or tumbling action on a flat or curved surface with the addition of a liquid.

Pelletizers can be classified based on the angle of their axis as horizontal drum or inclined dish pelletizers. Rotary fluidized granulators can also be used for pelletization. A standard fluidized drier bowl can be replaced with a rotating plate as an air distributor. For granulation, a binder liquid is sprayed from via one or two binary nozzles located axially to the rotational movement of the powder bed. This operation results in rounding of the granules to approximately spherical pellets. Such balling or agitation techniques can be influenced by operating conditions, such as bridging/binding liquid requirements, residence time of the material in the pelletizer, speed and angle of inclination of the pelletizer, amount of material fed to the pelletizer, choice and levels of binder, etc. One skilled in the art can readily adjust such factors to produce a satisfactory product.

The components of the invention can also be self binding. Liquid components can be pelletized with an the aid of suitable solidifying, binding or thickening agents.

The choice of an appropriate binder for a given application is readily determined by one skilled in the art. At a minimum, the binder must be capable of wetting the surfaces of the particle being pelletized or granulated. Binders must have sufficient wet strength to allow agglomerates to be handled, and sufficient dry strength to make them suitable for their intended purposes. Each process, however, makes use of a different system of forces and may require a different agglomerate strength. The final selection of the binder should be made on the basis of the type of equipment that is used. The size and size distribution of pellets, bulk density, strength and flow properties also affect the performance of the pellets, and these properties can be adjusted by one skilled in the art by the inclusion of additives, choice of equipment, and processing conditions.

8.2. Extrusion

Extrusion is a well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders.

Encapsulation by Extrusion: In this method, the lipid composition in the form of an emulsion is added to a low moisture melt of low maltodextrin, or sugar, or modified edible starch, mixed and extruded into a cold bath. The solidified composition can be further ground down. Optionally, centrifugal extrusion can be utilized for efficiency.

Melt Extrusion: Components of the invention can be melted and extruded with a continuous, solvent free extrusion process, with or without inclusion of additives. Such a process is well-established and well-known to skilled practitioners in the art.

8.3. Spheronization

Spheronization is the process of converting material into spheres, the shape with the lowest surface area to volume ratio. Spheronization typically begins with damp extruded particles. The extruded particles are broken into uniform lengths instantaneously and gradually transformed into spherical shapes. In addition, powdered raw materials, which require addition of either liquid or material from a mixer, can be processed in an air-assisted spheronizer.

8.4. Disk Spinning

Spinning disk technology involves formation of particles from either a molten or solubilized composition sprayed onto a rotating disc maintained at a predetermined temperature and rotating at a predetermined speed. When a molten composition contacts the spinning disk, particles congeal, while with a liquid composition, the liquid rapidly evaporates from the solution resulting in formation of solid drug particles. The composition may or may not contain various inert excipients and additives in addition to the active agent or agents. As will be appreciated by those of ordinary skill in the art, the temperature of the disc and its rotation may be adjusted to produce particles of predetermined size and shape.

8.5. Spray Congealing (Spray Chilling or Prilling)

Spray congealing is method that is generally used in changing the structure of the materials, to obtain free flowing powders from liquids and to provide pellets ranging in size from about 0.25 to 2.0 mm. Spray congealing is process in which a substance of interest is allowed to melt, disperse, or dissolve in a hot melt of other additives, and is then sprayed into an air chamber wherein the temperature is below the melting point of the formulation components, to provide spherical congealed pellets. The air removes the latent heat of fusion. The temperature of the cooled air used depends on the freezing point of the product. The particles are held together by solid bonds formed from the congealed melts. Due to the absence of solvent evaporation in most spray congealing processes, the particles are generally non porous and strong, and remain intact upon agitation. The characteristics of the final congealed product depend in part on the properties of the additives used. The rate of feeding and inlet/outlet temperatures are adjusted to ensure congealing of the atomized liquid droplet. The feed should have adequate viscosity to ensure homogeneity. The conversion of molten feed into powder is a single, continuous step. Proper atomization and a controlled cooling rate are critical to obtain high surface area, uniform and homogeneous congealed pellets. Adjustment of these parameters is readily achieved by one skilled in the art.

The spray congealing method is particularly suitable for heat labile substances, since ambient temperature is used to dry, and for moisture sensitive substances, since non-aqueous compositions can be utilized. Spray congealing is similar to spray drying, except that no solvent is utilized. Spray congealing is a uniform and rapid process, and is completed before the product comes in contact with any equipment surface. Most additives that are solid at room temperature and melt without decomposition are suitable for this method.

Conventional spray dryers operating with cool inlet air have been used for spray congealing. Several methods of atomization of molten mass can be employed, such as pressure, or pneumatic or centrifugal atomization. For persons skilled in the spray congealing art, it is well known that several formulation aspects, such as matrix materials, viscosity, and processing factors, such as temperature, atomization and cooling rate affect the quality (morphology, particle size distribution, polymophism and dissolution characteristics) of spray congealed pellets. The spray congealed particles may be used in tablet granulation form, encapsulation form, or can be incorporated into a liquid suspension form.

8.6. Solvent Dehydration (Spray Drying)

For compositions that are oily in nature, the spray drying technique is commonly employed. The oily material is commonly mixed with a polymeric material, such as gelatin, vegetable gum, modified starch, dextrin, or other appropriate additives. An emulsifier is added, if needed, to form an oil-in-water emulsion. The emulsion is atomized into a column of heated air in a drying chamber, resulting in rapid evaporation of water. Alternatively, the emulsion is atomized directly into a polar solvent, such as isopropanol, ethanol, glycerol or polyglycols, to dehydrate the aerosolized particle. This method is particularly suitable for compositions containing lipophilic actives or additives that result in lipophilic cores. Spray drying/solvent dehydration can also be applied to hydrophilic active ingredients or additives to form an oil in water emulsion which is spray dried. This results in a homogenous solid composition. Furthermore, water or organic solvent based formulations can be spray dried by using inert process gas, such as nitrogen, argon and the like.

8.7. Crystallization

Components of the present invention can be dissolved in appropriate solvents and subjected to spherical crystallization techniques well-known in the art.

8.8. Nanoencapsulation

Nanoencapsulation involves solubilizing an aqueous solution of an active ingredient and other components in a weakly polar vehicle. Micelles are formed with the active in an organic outer phase. Then, an amphiphilic monomer is added to the lipophilic external phase. The mixed micelles thus formed are then polymerized with the aid of a suitable procedure, such as UV or gamma radiation, heat, or chemical agents. the hardened solidified micelles are made to undergo phase exchange by replacing an outer lipophilic vehicle by water. By selecting appropriate monomers, networking agents and auxiliary materials, nanoncapsules as small as 80 to 250 nm can be prepared.

8.9. Precipitation by Anti-solvent

A solution of active agent and optionally other excipients in a solvent, preferably a volatile one, is mixed with an anti-solvent that has significantly lower solubility for the formulation components than does the solvent. As a result, the active agent precipitates out, together with excipients if present. The precipitate can be collected and subjected to further processes, such as size reduction. A pure precipitate of the active agent can be obtained from a solvent if no excipients are present.

8.10. Supercritical Fluid Processes

Components of the present invention can be dispersed in a supercritical fluid and crystallized as needed. Current techniques involving supercritical fluids include precipitation by rapid expansion of supercritical solutions, gas anti-solvent processes, and precipitation from gas saturated solutions.

8.1 1. Coacervation

Coacervation is a transfer of macromolecules with film properties from a solvated state in a coacervation phase into a phase in which there is a film around each particle. The coacervation method involves dispersing the composition in a dispersion of a polymeric colloid, such as gelatin alginate, and shock treating the mixture with temperature or pH, etc., to generate a two-phase system. The desired phase is then hardened with a cross-linking agent, such as glutaraldehyde.

8.12. Cryopelletization

The cryopelletization procedure allows conversion of a molten mass, aqueous solution or suspension into solid, bead-like particles. The molten mass solutions or suspensions are dripped by means of an appropriately designed device into liquid nitrogen. The production of small drops and liquid nitrogen cooling permit very rapid and uniform freezing of the material processed. The pellets are further dried in conventional freeze dryers. Cryopelletization can also be carried out under aseptic conditions for sterile processing. The most critical step producing spherical particles by globulization is the droplet formation. Droplet formation is influenced by formulation related variables, such as the nature of the active ingredient and additives, viscosity, total solid content, surface tension, etc. Extra care must be undertaken with processing of suspensions to ensure homogeneity. In addition, equipment design and processing variable also play an important role. One skilled in the art can readily balance the various factors to produce a satisfactory product. Enteric matrix pellets can be formed that include polyacrylic acid (e.g. Carbopol) with a high molecular weight polyethylene (such as PEG-20,000).

Other processes suitable for producing solid compositions of the pharmaceutical compositions of the present invention include extrusion and spray chilling. These processes are described in detail in Oshlack, et al., U.S. Pat. No. 5,965,161 and Leonard, U.S. Pat. No. 5,539,000 respectively, the disclosures of which are incorporated herein by reference.

For processing of encapsulated compositions, various methods can be used. The term “microencapsulation” applies to enclosure or encasement in microcapsules. Microencapsulation is a means of applying coatings to small particles of solids or droplets of liquids and dispersions. The terms “coated”, “protected” or “layered” are commonly used interchangeably with the term “encapsulated”. All of these terms can be used to refer to practically any core material that is encased or enclosed in an outer shell. Typical equipment used to apply coating includes a conventional pan (Pellegrini; Italy), a modified perforated pan (multicoater, Thomas Eng., Ill.) or a Wurster coater in a Glatt powder doater/granulator (Glatt Airtechniques).

8.13. Solvent Based Solution Coating

Solvent-based coating is when the components of the invention are solubilized and/or dispersed in a solvent. The solvent can be aqueous. When the solvent is aqueous-based, the components can be emulsified with an appropriate emulsifier, organic solvent, or a supercritical fluid. Solvents with a lower melting point than water and higher evaporation numbers are preferred. Solvent mixtures with other organic solvents or water are often employed to get appropriate viscosity and component solubilization. Typical solvents include ethanol, methanol, isopropanol, acetone, dichloromethane, trichloromethane and ethyl acetate. Appropriate polymers can also be added as needed. Cellulosic derivatives and polymethacrylates are particularly suitable additives for organic solvent coating. Dissolution and solubilization of the components is facilitated by rigorous stirring or heating. Plasticizers may be also be added to stimulate dissolution. Colorants and antisticking agents can be employed as needed.

Substrate surface area, shape, porosity and stability are important determinants of good coating. Spherical particles are preferred, and these may be produced through spheronization or a spherical crystallization process. Crystals or compact granules from dry compaction or extrusion processes, often available commercially, serve as good substrates.

Encapsulation can be conducted by traditional pan coating or fluidized bed techniques. Several process (air supply, temperature, spray rate, spray system, powder feed, attrition) and formulation factors determine the quality of the end product, and one skilled in the art can readily adjust such parameters as needed.

Air suspension in a rotary fluidized bed granulator can used to deposit the encapsulation coat on to a substrate, thus allowing a high rate of drug application with low drug loss. Furthermore, both aqueous and organic solvents can be used. The Wurster process, an air suspension technique, is more suitable for encapsulations involving very fine powders.

8.14. Solvent-Free Coating

This process entails using coating materials that can be applied in a molten state. The selection of proper coating materials depends on melting point, melting point range and the viscosity in the liquid state. A fluidized bed is ideal for molten coatings of substrates that range from about 100-2000 microns in size. Fluidized bed coating, spraying molten materials, involves achieving a proper balance of process parameters that allow proper encapsulation to occur. Substrate particles that are suspended and separated from each other by the fluidization air enter a zone of finely atomized coating liquid. Coating occurs as the liquid droplets, which are substantially smaller in size than substrate, impact the particles, spread, and solidify. Multiple layers can be coated, and the completion of spraying is followed by a product stabilization or cooling step. Some critical success parameters include bed temperature, atomization, atomization fluid temperature, or droplet size, spray type, spray rate, rate of coating droplet solidification on particle surfaces, particle size, shape, etc. Inert materials such as sodium chloride, citric acid, potassium chloride can serve as substrates. One skilled in the art can readily adjust such parameters to achieve a satisfactory product.

The processes described above are suitable for treating substrate-based compositions or non-substrate-based compositions of the present invention. Thus, in one embodiment, pharmaceutical compositions of the present invention do not include a seed particle, such as a conventional drug or other additive aggregate starch or sugar bead. Instead, the compositions are processed, and the components are chosen, such that a solid composition is formed without the need to coat the composition onto a substrate bead. Such compositions can be in the form of beadlets, beads, granules, pellets, etc., that have an approximately homogenous distribution of active ingredient, surfactant, triglyceride and/or additives. These compositions can be produced by means of balling in pelletizers or fluid bed granulators, and compaction or extrusion/spheronization. In addition, these compositions can be produced using solvent-free spray congealing processes or dropping (globulization) methods. Dropping procedures involve conversion of aqueous solutions or suspensions to a solid form. Congealing of the liquid droplets in cooling baths can aided by a chemical reaction (e.g., insoluble salt or complex formation), a sol/gel transition, or by freezing in a coolant bath of liquid nitrogen or halogenated hydrocarbons.

9. Specific Formulations

In one embodiment, the pharmaceutical composition includes a solid carrier that is an admixture of an active ingredient and at least one of the following: a hydrophilic surfactant, a lipophilic component such as a lipophilic surfactant or a triglyceride, or a solubilizer. In some embodiments of the invention, this matrix type formulation may be preferred over the substrate-containing (inert core or active containing core) formulation with an encapsulation coat, as this enables a higher drug loading per dosage form, thus resulting in a potentially smaller dosage size. Preferably, the admixture of the solid carrier is substantially hydrophilic, including an active ingredient, and at least one hydrophilic surfactant and/or at least one hydrophilic solubilizer. In one particularly preferred embodiment, the solid carrier is prepared by a spray congealing (spray chill or prilling) process so that no water or organic solvents are introduced, the presence of which can be detrimental to the stability of certain active ingredients.

In another embodiment, the solid pharmaceutical composition includes a solid carrier, the solid carrier including a substrate and an encapsulation coat on the substrate. The encapsulation coat includes an active ingredient and at least one of the following: a hydrophilic surfactant, a lipophilic component such as a lipophilic surfactant or a triglyceride, or a solubilizer. Preferably, the encapsulation coat is substantially hydrophilic, includes an active ingredient and at least one hydrophilic surfactant and/or at least one hydrophilic solubilizer.

Prior art has used surfactants in formulating coated bead compositions to provide a wetting function, to enable hydrophobic drugs to properly adhere to beads and/or water-soluble binders. For example, U.S. Pat. No. 4,717,569 to Harrison et al. discloses coated bead compositions of hydrophobic steroid compounds wetted by a hydrophilic surfactant and adhered to the beads by a water-soluble binder. The steroid compound is present as finely divided particles, held to the beads by the binder. The present inventors have surprisingly found that proper choice of surfactants and other components allows compositions to be prepared with a wide variety of active ingredients. For example, while the Harrison reference discloses the use of surfactants as wetting agents, the present inventors have found that surfactants at higher levels, i.e., in amounts far in excess of the amounts necessary or appropriate for a wetting function, enable a pharmaceutical active ingredient to be fully or at least partially solubilized in the encapsulation coating material itself, rather than merely physically bound in a binder matrix. In fact, while binders can optionally be used in the compositions of the present invention, the higher surfactant concentrations of the present invention, i.e., solubilizing amounts, obviate the need for binders and render them optional instead of necessary.

The amount of hydrophilic surfactant used in this embodiment can be adjusted so as to at least partially or fully solubilize the pharmaceutical active ingredient, with the optional lipophilic surfactants, triglycerides and solubilizer chosen to further increase the pharmaceutical active ingredient's solubility. In one embodiment of the invention, the hydrophilic surfactant comprises about 1-95 wt %, and preferably about 15-75 wt % of the admixture or the encapsulation coat of the solid carrier. More preferably, the hydrophilic surfactant comprises at least 10 wt % of the admixture or the encapsulation coat of the solid carrier.

The lipophilic additive (lipophilic surfactant or triglyceride) can be used as desired to further enhance solubilization of the active ingredient, or to promote dispersion (emulsification/micellization) in vivo, or to promote in vivo absorption at the absorption site. In one embodiment of the invention, the lipophilic additive comprises about 1-95 wt %, and preferably about 20-80 wt % of the formulation.

The solubilizer can be used as desired to further enhance solubilization of the active ingredient, or to promote dispersion (emulsification/micellization) in vivo, or to promote in vivo absorption at the absorption site. For an immediate-release dosage form, the solubilizer is present such that no less than 80% of the active ingredient is released in about 5-60 minutes after the dosage reaches the small intestine which can be evaluated according to the USP XXII dissolution test (type 1, basket) at 100 rpm, in simulated intestinal fluid (SIF, a phosphate buffer medium, at pH 6.8) or in simulated gastric fluid (SGF). For a sustained-release dosage form, the solubilizer is present such that at least 80% of the active ingredient is released in about 2-8 hours in a linear or pulsatile fashion. In one embodiment of the invention, the solubilizer comprises about 0.1-95 wt %, and preferably 0.25-80 wt % of the formulation.

A further advantage believed to accrue from the pharmaceutical compositions of the present invention is that upon administration of the composition to a patient, the high levels of surfactants and other components present in the composition facilitate the rapid solubilization of the pharmaceutical active ingredient. Thus, while the prior art composition of Harrison contains a drug in a form which requires further solubilization in vivo, such as by emulsification and micellization in the gastrointestinal tract, the active ingredient in compositions of the present invention is at least partially solubilized in the composition itself, and is further provided with surfactants and other components in the composition to facilitate rapid dispersion (emulsification/micellization) and sustained solubilization of the active ingredient upon administration.

It should be noted that in this embodiment, the encapsulation coat can alternatively be formulated without the active ingredient. In this aspect, an active ingredient can be provided in the composition itself but not in the encapsulation coat, if desired. While not presently preferred, such a formulation delivers the active ingredient to the patient along with the surfactants or other components to facilitate dispersion (emulsification/micellization), thus still providing more rapid active ingredient presentation to the absorption site. Alternatively, the active ingredient can be administered in a separate dosage form, including a conventional dosage form, prior to, concurrently with, or subsequent to administration of the present compositions, to achieve similar advantages.

For more hydrophilic active ingredients, the materials of the encapsulation coat provides components to promote efficient transport of the active ingredient across the barrier membrane to promote more effective absorption. For these active ingredients, it is preferable to include a lipophilic component in the encapsulation coat.

In another embodiment, the solid pharmaceutical composition includes a solid carrier, the solid carrier including a substrate and an encapsulation coat on the substrate. The encapsulation coat includes a hydrophilic surfactant. Optionally, the encapsulation coat can include a pharmaceutical active ingredient, a lipophilic additive, a solubilizer, or both an active ingredient and a lipophilic additive and/or a solubilizer.

In another embodiment, the solid pharmaceutical composition includes a solid carrier, the solid carrier including a substrate and an encapsulation coat on the substrate. The encapsulation coat includes a lipophilic component, such as a lipophilic surfactant or a triglyceride. Optionally, the encapsulation coat can include a pharmaceutical active ingredient, an ionic or non-ionic hydrophilic surfactant, a solubilizer, or both an active ingredient and a hydrophilic surfactant and/or a solubilizer. In this embodiment, the lipophilic surfactant or triglyceride can be present in amounts to enable at least partial solubilization of an active ingredient in the encapsulation coat, in the composition, or separately administered.

In another embodiment, the solid pharmaceutical composition effectively presents a lipophilic component with or without an active ingredient to help promote absorption of a hydrophilic active.

In another embodiment, the solid pharmaceutical composition includes a solid carrier, the solid carrier including a substrate and an encapsulation coat on the substrate. The encapsulation coat includes at least one solubilizer. The encapsulation coat can also include a pharmaceutical active ingredient, a lipophilic component such as a lipophilic surfactant or a triglyceride, or a hydrophilic surfactant. The encapsulation coat can also contain an active ingredient, and a lipophilic component and/or or hydrophilic surfactant.

In another embodiment, the solid pharmaceutical composition includes a solid carrier, the solid carrier including a substrate and an encapsulation coat on the substrate. The encapsulation coat includes an active ingredient and an ionic or non-ionic hydrophilic surfactant; an active ingredient and a lipophilic component such as a lipophilic surfactant or a triglyceride; an active ingredient and a solubilizer; or an active ingredient and any combination of hydrophilic surfactants, lipophilic components and solubilizers.

In another embodiment, the solid pharmaceutical composition includes a solid carrier, wherein the solid carrier is formed of at least two components selected from the group consisting of pharmaceutical active ingredients; ionic or non-ionic hydrophilic surfactants; lipophilic components such as lipophilic surfactants and triglycerides; and solubilizers. For example, the solid carrier can be comprised of the active ingredient, a hydrophilic surfactant and a lipophilic additive, where the weight ratio of lipophilic additive to the hydrophilic surfactant is in the range of about 0.10:1 to 0.92:1.

In this embodiment, the solid pharmaceutical composition is formulated without the need for a substrate seed particle. The active ingredient, surfactants, triglycerides and solubilizers in the chosen combination are processed, with appropriate excipients if necessary, to form solid carriers in the absence of a seed substrate. Preferably, the components are chosen to at least partially solubilize the active ingredient, as described above.

10. Methods

The present invention also provides methods of using the above-described pharmaceutical composition. In one aspect, the present invention provides a method of treating a patient with an active ingredient, the method including the steps of providing a dosage form of a pharmaceutical composition as described above, including an active ingredient, and administering the dosage form to the patient. The patient can be an animal, preferably a mammal, and more preferably a human.

In another aspect, the present invention provides a method including the steps of providing a dosage form of a pharmaceutical composition as described above, providing a dosage form of an active ingredient, and administering the dosage forms to the patient. This method is advantageous when all or part of the active ingredient or an additional active ingredient is to be administered to the patient in a separate dosage form prior to, concurrently with, or subsequent to administration of the pharmaceutical composition.

In another aspect, the present invention provides a method of improving the palatability and/or masking the taste of an active ingredient, by providing the active ingredient in a pharmaceutical composition as described above. Since the active ingredient is encapsulated in a lipid coat, it will not instantaneously dissolve in the mouth, but will instead dissolve in a region past the oral cavity, thereby substantially avoiding or at least reducing undesirable contact between the active ingredient and the mouth.

In yet another aspect, the invention provides for orally administering a dosage form of the pharmaceutical composition as described above, for the treatment of individuals that have one or more of the following conditions: duodenal ulcer, gastric ulcer, peptic ulcer (any H. pylori negative or H. pylori positive peptic ulcer), gastroesophageal reflux disease (GERD or GORD with or without esophagitis), erosive esophagitis, Barrett's esophagus, NSAID-related gastrointestinal complications, H. pylori infection, acid-related dyspepsia, and pathological hypersecretory conditions (including Zollinger-Ellison syndrome). The invention also provides for orally administering a dosage form of the pharmaceutical composition as described above, for the management of any acid-related disorders including initial healing and maintenance therapy to prevent the disorders from reoccurring.

The active ingredient can be a proton pump inhibitor such as lansoprazole, omeprazole, esomeprazole, pantoprazole, rabeprazole, as well as a pharmaceutically acceptable salt, isomer or derivative thereof.

Exemplary dosing regiments for lansoprazole include 15 mg/day for short-term treatment of duodenal ulcers, for maintenance of healed duodenal ulcers, for short-term treatment of symptomatic GERD, and for maintenance of healing of erosive esophagitis; and 30 mg/day for short-term treatment of gastric ulcers and short-term treatment of erosive esophagitis.

Exemplary combined dosing regiments for lansoprazole, amoxicillin, and clarithromycin for H. pylori eradication to reduce the risk of duodenal ulcer recurrence, is a twice daily dose of 30 mg lansoprazole, 1 g amoxicillin, and 500 mg clarithromycin. Exemplary combined dosing regiments for lansoprazole and amoxicillin for the same indication, is a thrice daily dose of 30 mg lansoprazole and 1 g amoxicillin.

Exemplary dosing regiments for rabeprazole include 20 mg/day for healing and maintenance of healing of GERD and healing of duodenal ulcers; and 60-100 mg/day for treatment of pathological hypersecretory conditions including Zollinger-Ellison Syndrome.

Exemplary dosing regiments for pantoprazole include 10-40 mg/day for treatment of erosive esophagitis associated with GERD.

Exemplary dosing regiments for omeprazole include 20 mg/day for short-term treatment of active duodenal ulcers, for treatment of symptomatic GERD, and for maintenance of healing of erosive esophagitis; and 40 mg/day for treatment of gastric ulcers.

Exemplary combined dosing regiments for omeprazole, amoxicillin, and clarithromycin for H. pylori eradication to reduce the risk of duodenal ulcer recurrence, is a twice daily dose of 20 mg omeprazole, 500 mg amoxicillin, and 1000 mg clarithromycin. Exemplary combined dosing regiments for omeprazole and amoxicillin for the same indication, is a once daily dose of 40 mg omeprazole and 500 mg clarithromycin t.i.d.

Exemplary dosing regiments for esomeprazole include 20-40 mg/day for healing and maintenance of healing of erosive esophagitis and for treatment of symptomatic gastroesophageal reflux disease.

Exemplary combined dosing regiments for esomeprazole, amoxicillin, and clarithromycin for H. pylori eradication to reduce the risk of duodenal ulcer recurrence, is a once daily dose of 40 mg esomeprazole and twice daily dose of 1000 mg amoxicillin, and 500 mg clarithromycin.

In another aspect of the invention, the compositions enable gastric resistance or acid degradation reduction of the active ingredient. This enables improved delivery of an active agent that is acid-labile, as such compounds are particularly prone to or susceptible to acid degradation. More specifically, the compositions of the invention provide for an improved in vivo and ex vivo stability of active ingredients in environments having a pH within the range of about 1-6.8. This improved stability is in comparison to currently available dosage forms, and the formulations of the invention are expected to provide up to 50-100% improvement in the in vivo and ex vivo stability of active ingredients such as acid-labile drugs, preferably higher. The compositions of the invention are robust in performance due to low release and/or degradation in the acidic environment of the stomach, and rapid release of active ingredient in the small intestine (duodenum). This can be achieved through traditional enteric coating or by a hydrophobic digestible seal coat or through a simple matrix system containing a moisture repelling material such as a hydrophilic surfactant or lipophilic additive. The unique compositions of this invention provide a dosage from that rapidly releases the active ingredient in the duodenum due to digestion/solubilization of the core/coat aided by enzymes, bile secretion, etc. The dosage form further protects the drug upon release prior to absorption, unlike traditional compositions, through efficient emulsification/micellization of excipients and partitioning of drug in such phases to protect the drug from degradation, especially in mildly acidic conditions.

In another aspect of the invention, the solid carrier improves the chemical stability of the active ingredient. This encompasses both in vivo and ex vivo stability under acidic conditions as noted above, as well as during storage when the carrier may inadvertently be exposed to moisture (typical ambient conditions with proper packaging). The improved stability under storage conditions with respect to the potency and/or the discoloration of the dosage form is in comparison to a dosage form wherein the active ingredient not evenly in contact with a base. The in vivo or ex vivo stability of the active ingredient under acidic condition is improved, such that the formulations of the invention are expected to provide up to 50-100% improvement in the half-life of active ingredients such as acid-labile drugs, and preferably higher. The stability of the active ingredient during storage can also improved by the inclusion of a lipophilic additive or seal coat, both of which can serve to reduce the permeation of moisture to the active ingredient, as compared to formulations that do not have a lipophilic additive or seal coat.

In another aspect of the invention, the solid carrier protects the upper gastrointestinal tract from the adverse effects of the active ingredient.

In another aspect, the present invention provides a method of improving the dissolution and/or absorption of a pharmaceutical active ingredient, by administering the active ingredient in a composition as described above, or co-administering the active ingredient with a composition as described above.

In yet another aspect of the invention, the compositions described herein provide for improving the absorption and/or bioavailability of active ingredients. More specifically, the oral bioavailability of active ingredients is improved when administered to mammals under fed condition, i.e., the state typically induced by the presence of food in the stomach. Once this fed mode has been induced, larger particles of food are retained in the stomach for a longer period of time than smaller particles. It is believed that the compositions of the invention will provide higher bioavailability resulting in superior performance and being less susceptible to food effects. This improved bioavailability is in comparison to currently available dosage forms, and the formulations of the invention are expected to provide up to 50-100% improvement in the oral bioavailability of active ingredients such as acid-labile drugs, and preferably higher. For example, without wishing to be bound by theory, it is believed that current formulations of proton pump inhibitors often experience a decrease in bioavailability of up to 50% when taken with food. This effect is likely due to the lowered duodenal pH upon gastric emptying and/or higher gastric retention that leads to drug instability. It is believed that compositions of the invention will not experience these shortcomings.

### EXAMPLES

### Example 1

### Preparation of Coated Beads

Compositions according to the present invention were prepared as follows. The specific components used are detailed in Examples 2-5.

A spraying solution of the coating materials was prepared by dissolving the desired amount of the active ingredient and mixing with the hydrophilic and/or lipophilic surfactants in an organic solvent or a mixture of organic solvents. The organic solvent used for the coating solution was a mixture of methylene chloride and isopropyl alcohol in a 3:1 to 1:1 weight ratio.

Commercially available sugar beads (30/35 mesh size) were coated in a conventional coating pan having a spray gun (Campbell Hausfield, DH 7500) with a nozzle diameter of 1.2 mm and an air pressure of 25 psi. The bed temperature was maintained at approximately 32° C. during the spraying process. Appropriate amounts of talc were sprinkled on the beads during the spraying process to reduce the agglomeration of coated beads. When the spraying process was completed, the coated beads were allowed to cool to room temperature. The coated beads were then dried under vacuum to minimize residual solvent levels. The dried, coated beads were then sieved and collected.

### Example 2

### Composition I

A pharmaceutical composition was prepared according to the method of Example 1, having a substrate particle, an active ingredient (glyburide), and a mixture of a hydrophilic surfactant (PEG-40 stearate) and a lipophilic surfactant (glycerol monolaurate). The components and their amounts were as follows:

| Component | Weight (g) | % (w/w) |
|---|---|---|
| Glyburide | 1 | 0.8 |
| PEG-40 stearate | 33 | 25.2 |
| Glycerol monolaurate | 17 | 13.0 |
| Nonpareil seed (30/35 mesh) | 80 | 61.1 |

### Example 3

### Composition II

A pharmaceutical composition was prepared according to the method of Example 1, having a substrate particle, an active ingredient (progesterone), a mixture of a hydrophilic surfactant (Solulan C-24) and two lipophilic components (deoxycholic acid and distilled monoglycerides). The components and their amounts were as follows:

| Component | Weight (g) | % (w/w) |
|---|---|---|
| Progesterone | 12 | 8.6 |
| Solulan C-24 (Amerchol)* | 32 | 22.9 |
| Distilled monoglycerides | 8 | 5.7 |
| Deoxycholic acid | 8 | 5.7 |
| Nonpareil seed (30/35 mesh) | 80 | 57.1 |

*PEG-24 cholesterol ether

### Example 4

### Composition III

A pharmaceutical composition was prepared according to the method of Example 1, having a substrate particle, an active ingredient (itraconazole), a mixture of non-ionic hydrophilic surfactants (Cremophor RH-40 and PEG-150 monostearate), an ionic hydrophilic surfactant (sodium taurocholate) and a lipophilic surfactant (glycerol monolaurate). The components and their amounts were as follows:

| Component | Weight (g) | % (w/w) |
|---|---|---|
| Itraconazole | 20 | 9.7 |
| Cremophor RH-40 (BASF)* | 25 | 12.1 |
| Glycerol monolaurate | 10 | 4.8 |
| Sodium taurocholate | 5 | 2.4 |
| PEG-150 monostearate | 27 | 13.0 |
| Nonpareil seed (30/35 mesh) | 120 | 58.0 |

*PEG-40 hydrogenated castor oil

### Example 5

### Composition IV

A pharmaceutical composition was prepared according to the method of Example 1, having a substrate particle, an active ingredient (omeprazole), a mixture of a two hydrophilic surfactants (PEG-150 monostearate and PEG-40 monostearate), and a triglyceride-containing lipophilic component (Maisine 35-1). The components and their amounts were as follows:

| Component | Weight (g) | % (w/w) |
|---|---|---|
| Omeprazole | 16 | 8.8 |
| PEG-150 monostearate | 50.4 | 27.8 |
| PEG-40 monostearate | 25.2 | 13.9 |
| Maisine 35-1 (Gattefosse)* | 8.4 | 4.6 |
| Magnesium carbonate | 1.6 | 0.9 |
| Nonpareil seed (30/35 mesh) | 80 | 44.1 |

*linoleic glycerides

### Example 6

### Seal Coating

The dried, coated beads of Example 3 were further seal coated by a polymer layer. The seal coating polymer layer was applied to the progesterone-coated beads in a Uni-Glatt fluid bed coater. Polyvinylpyrrolidone (PVP-K30) was dissolved in isopropyl alcohol to form a 5% w/w solution. This seal coating solution was sprayed onto the coated beads with a Wurster bottom spray insert. The inlet and outlet air temperature were maintained at 30 and 40° C., respectively. When the spraying process was complete, the beads were further dried by supplying dry air at 50-55° C. for 5-15 minutes. The seal coated beads were then allowed to cool in the apparatus by supplying dry air at 20-25° C. for 5-15 minutes. The dried, seal coated beads were collected and stored in a container.

### Example 7

### Protective Coating

The dried, coated beads of Example 5 were further coated with a protective polymer layer. The protective coating was applied to the omeprazole coated beads by spraying with a hydroxypropyl methylcellulose (HPMC) solution. The protective coating HPMC solution was prepared by dissolving 6 grams of HPMC in ethanol. To this solution, methylene chloride was added followed by 2 mL of triethylcitrate as a plasticizer and 1 g of talc. the HPMC solution was sprayed on the beads as described in Example 6, and the protective coated beads were then dried and collected.

### Example 8

### Enteric Coating

The dried, coated beads of Example 5 were further coated with an enteric coating layer. The enteric layer was applied to the omeprazole coated beads by spraying a Eudragit L100 solution. Eudragit L100 is an acrylate polymer commercially available from Rohm Pharma. The spraying solution was prepared by dispersing 15 g of Eudragit L100 in 200 mL of ethanol to give a clear solution. To this solution, 4 g of triethyl citrate was then added as a plasticizer. 2 grams of purified talc was also added to the solution. The solution was then sprayed onto the beads, and the beads were dried, as described in Example 6.

### Example 9

### Comparative Dissolution Study I

A comparative dissolution study was performed on three forms of an active ingredient: the glyburide coated beads of Example 2, a commercially available glyburide product (Micronase®, available from Pharmacia & Upjohn), and the pure glyburide bulk drug. The dissolution study was performed using a USP dissolution type 2 apparatus. For each of the three forms, material equivalent to 5 mg of glyburide was used for each triplicated dissolution run in 500 mL of isotonic pH 7.4 phosphate buffer. The dissolution medium was maintained at 37° C. and constantly stirred at a speed of 100 rpm. The dissolution media were sampled at 15, 30, 45, 60, 120 and 180 minutes. At each time point, 3 mL of the medium was sampled, and the medium was replenished with 3 mL of fresh buffer. The samples were filtered through a 0.45 μ filter immediately after the sampling. The filtrates were then diluted in methanol to an appropriate concentration for a glyburide-specific HPLC assay.

The HPLC assay was performed on a Varian 9010 system by injecting 50 μL of the sample. The sample was separated on a Phenominex C1 8 column by running a mobile phase of 75:25 v/v methanol/phosphate buffer (0.1 M potassium dihydrogen phosphate, pH adjusted to 3.5 using phosphoric acid), at a flow rate of 1 mL/min, at ambient temperature. Glyburide was detected by its UV absorption at 229 nm.

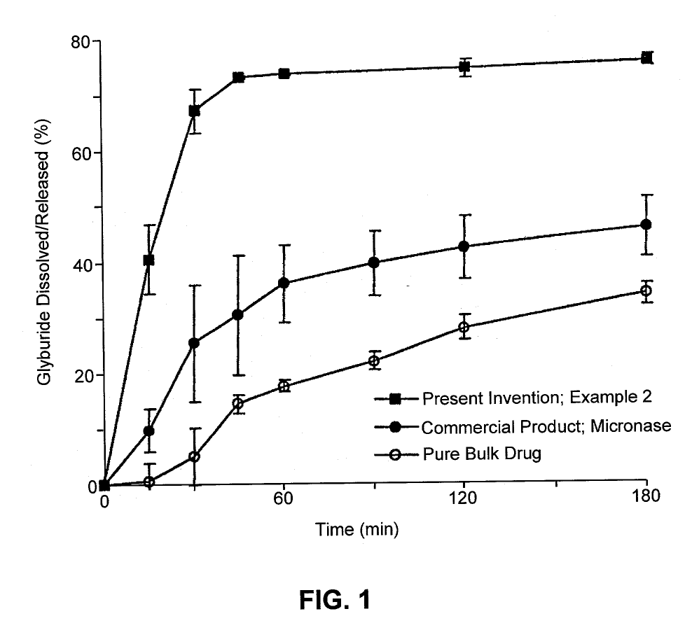

The results of the comparative dissolution measurement were expressed as the percent of glyburide dissolved/released in the dissolution medium at a given time, relative to the initial total glyburide content present in the dissolution medium (5 mg/500 mL). The results are shown in FIG. 1, with the error bars representing the standard deviation. As the Figure shows, the glyburide coated beads of the present invention showed a superior dissolution profile in the rate, the extent, and the variability of glyburide dissolved/released into the dissolution medium, compared to the commercial Micronase® and the pure bulk drug.

### Example 10

### Comparative Dissolution Study II

A comparative dissolution study was performed on three forms of an active ingredient: the progesterone coated beads of Example 3, the seal coated, progesterone coated beads of Example 6, and the pure progesterone bulk drug. The dissolution study was performed using a USP dissolution type 2 apparatus. For each of the three forms, material equivalent to 100 mg of progesterone was used for each duplicated dissolution run in 900 mL of isotonic pH 7.4 phosphate buffer containing 0.5% w/v of Tween 80. The dissolution medium was maintained at 37° C. and constantly stirred at a speed of 100 rpm. The dissolution media were sampled at 30, 60, 120 and 180 minutes. At each time point, 3 mL of the medium was sampled, and the medium was replenished with 3 mL of fresh buffer/Tween solution. The samples were filtered through a 0.45μ filter immediately after the sampling. The filtrates were then diluted in methanol to an appropriate concentration for a progesterone-specific HPLC assay.

The HPLC assay was performed on a Varian 9010 system by injecting 50 μL of the sample. The sample was separated on a Phenominex C18 column by running a mobile phase of 75:25 v/v methanol/phosphate buffer (0.1 M potassium dihydrogen phosphate, pH adjusted to 3.5 using phosphoric acid), at a flow rate of 1 mL/min, at ambient temperature. Glyburide was detected by its UV absorption at 229 nm.

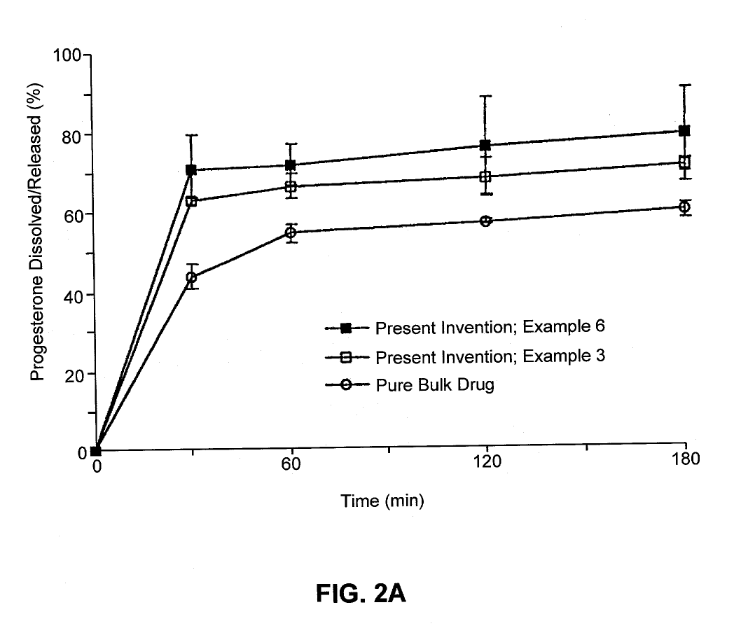

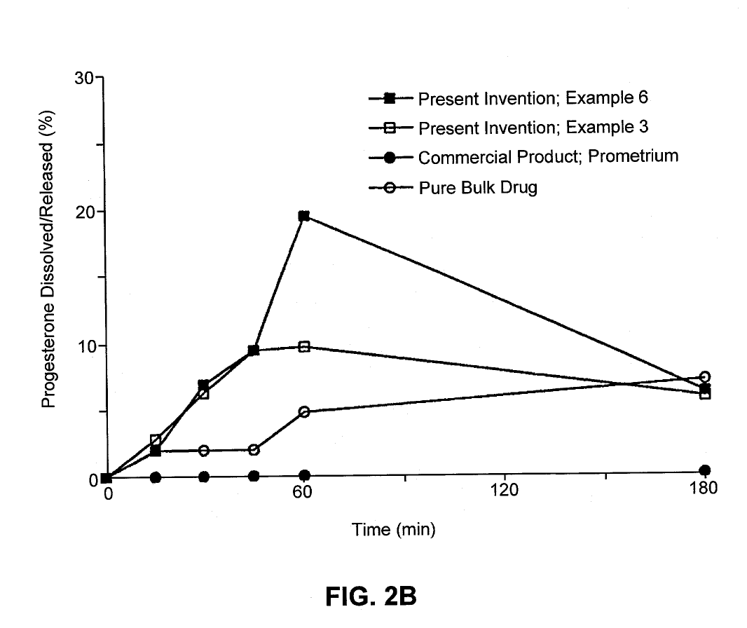

The results of the comparative dissolution measurement were expressed as the percent of progesterone dissolved/released in the dissolution medium at a given time, relative to the initial total progesterone content present in the dissolution medium (100 mg/900 mL). The results are shown in FIG. 2A. As the Figure shows, the progesterone coated beads of the present invention, with or without a seal coating, showed superior dissolution profiles in both the rate and the extent of progesterone dissolved/released into the dissolution medium, compared to the pure bulk drug.

### Example 11

### Comparative Dissolution Study III

A comparative dissolution study was performed on three forms of an active ingredient: the progesterone coated beads of Example 3, the seal coated, progesterone coated beads of Example 6, and the pure progesterone bulk drug. Prometrium® is a capsule dosage form in which micronized progesterone is suspended in a blend of vegetable oils. The dissolution of the Prometrium® capsule was performed using a USP dissolution type 1 apparatus, and the dissolution of the other samples was performed using a USP dissolution type 2 apparatus. For each of the three forms, material equivalent to 100 mg of progesterone was used for each dissolution run in 900 mL of isotonic pH 7.4 phosphate buffer. The dissolution medium was maintained at 37° C. and constantly stirred at a speed of 100 rpm. The dissolution media were sampled at 15, 30, 45, 60 and 180 minutes. At each time point, 3 mL of the medium was sampled, and the medium was replenished with 3 mL of fresh buffer/Tween solution. The samples were filtered through a 0.45μ filter immediately after the sampling. The filtrates were then diluted in methanol to an appropriate concentration for a progesterone-specific HPLC assay.

The HPLC assay was performed on a Varian 9010 system by injecting 50 μL of the sample. The sample was separated on a Phenominex C18 column by running a mobile phase of 75:25 v/v methanol/phosphate buffer (0.1 M potassium dihydrogen phosphate, pH adjusted to 3.5 using phosphoric acid), at a flow rate of 1 mL/min, at ambient temperature. Glyburide was detected by its UV absorption at 229 nm.

The results of the comparative dissolution measurement were expressed as the percent of progesterone dissolved/released in the dissolution medium at a given time, relative to the initial total progesterone content present in the dissolution medium (100 mg/900 mL). The results are shown in FIG. 2B. As the Figure shows, the progesterone coated beads of the present invention, with or without a seal coating, showed superior dissolution profiles in both the rate and the extent of progesterone dissolved/released into the dissolution medium, compared to the commercial Prometrium® and the pure bulk drug.

### Example 12

### Comparative Dissolution Study IV

A comparative dissolution study was performed comparing the rate and extent of dissolution of the protective coated, omeprazole coated beads of Example 7, the enteric coated, omeprazole coated beads of Example 8 and a commercially available omeprazole product (Prilosec®, available from Astra Zeneca). The dissolution study was performed using a USP dissolution type 2 apparatus. For each of the three dosage forms, material equivalent to 5 mg of omeprazole was used for each dissolution run in 500 mL of isotonic pH 7.4 phosphate buffer. The dissolution medium was maintained at 37° C. and constantly stirred at a speed of 100 rpm. The dissolution media were sampled at 15, 30, 45 and 60 minutes. At each time point, 3 mL of the medium was sampled, and the medium was replenished with 3 mL of fresh buffer. The samples were filtered through a 0.45μ filter immediately after the sampling. The filtrates were then diluted in methanol to an appropriate concentration for an omeprazole-specific HPLC assay.

The HPLC assay was performed on a Varian 9010 system by injecting 50 μL of the sample. The sample was separated on a Phenominex C18 column by running a mobile phase of 30:70 v/v acetonitrile/phosphate buffer (pH 7.4), at a flow rate of 1.1 mL/min, at ambient temperature. Omeprazole was detected by its UV absorption at 302 nm.

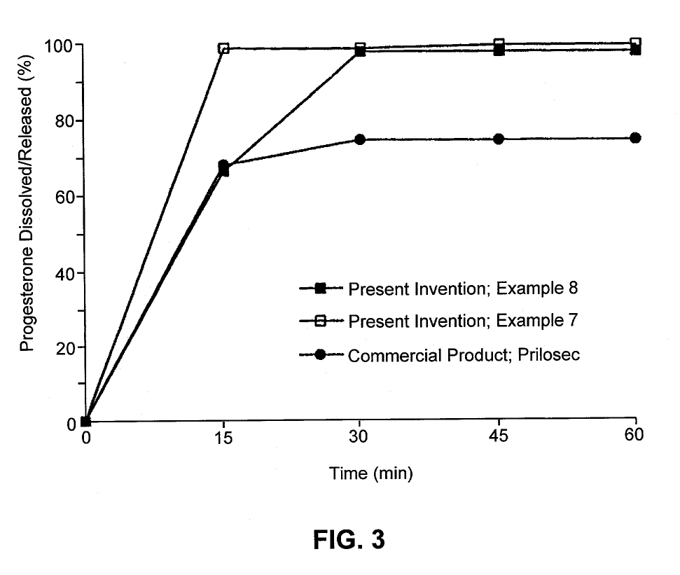

The results of the comparative dissolution measurement were expressed as the percent of omeprazole dissolved in the dissolution medium at a given time, relative to the initial total omeprazole content present in the dissolution medium (5 mg/500 mL). The results are shown in FIG. 3. As the Figure shows, the omeprazole coated beads of the present invention showed superior dissolution profiles in both the rate and the extent of omeprazole dissolved/released into the dissolution medium, compared to the commercial Prilose® product.

The following non-limiting Examples 13-28 illustrate compositions that can be prepared according to the present invention. It should be appreciated that the compositions can be prepared in the absence of the active ingredients and appropriate amounts of the active ingredients in any given dosage form then can be administered together or separately with the composition. It should also be appreciated that the compositions can further include additional additives, excipients, and other components for the purpose of facilitating the processes involving the preparation of the composition or the pharmaceutical dosage form, as described herein, as is well-known to those skilled in the art.

### Example 13

| Component | Amount (g) |
|---|---|
| Atorvastatin | 4 |
| Partially hydrogenated soybean oil | 10 |
| Myrj 52 (PEG-40 stearate) | 70 |
| Monomuls 90-45 (glyceryl monolaurate) | 20 |
| Nonpareil seed (25/30 mesh) | 120 |

### Example 14

| Component | Amount (g) |
|---|---|
| Alendronate sodium | 50 |
| Cremophor RH-40 (PEG-40 hydrogenated | 100 |
| castor oil) | |
| Capmul MCM (glyceryl | 50 |
| caprylate/caprate) | |
| Sodium alginate | 2 |
| Water | 5 |
| Nonpareil seed (25/30 mesh) | 200 |

### Example 15

| Component | Amount (g) |
|---|---|
| Ganciclovir | 100 |
| Tocopheryl PEG-1000 succinate | 200 |
| lmwitor 191 (glyceryl monostearate) | 30 |
| Water | 20 |
| Nonpareil seed (25/30 mesh) | 400 |

### Example 16

| Component | Amount (g) |
|---|---|
| Simvastatin | 20 |
| Hydrogenated castor oil | 40 |
| Crodet O40 (PEG-40 oleate) | 200 |

### Example 17

| Component | Amount (g) |
|---|---|
| Zafirlukast | 7 |
| PEG-150 monostearate | 50 |
| PEG-40 monostearate | 80 |
| Peceol (glyceryl monooleate) | 15 |

### Example 18

| Component | Amount | |
|---|---|---|
| Salmon calcitonin | 300,000 | IU |
| PEG-40 monostearate | 200 | g |
| Glycerol monolaurate | 100 | g |
| Water | 5 | g |

### Example 19

| Component | Amount (g) |
|---|---|
| Lovastatin | 20 |
| Coenzyme Q10 | 50 |
| PEG-40 stearate | 150 |
| Glycerol monolaurate | 50 |
| Nonpareil seed (25/30 mesh) | 200 |

### Example 20

| Component | Amount (g) |
|---|---|
| Tacrolimus | 5 |
| Solulan C-24 | 130 |
| Distilled monoglycerides | 40 |
| Deoxycholic acid | 80 |
| Nonpareil seed (35/40 mesh) | 250 |

### Example 21

| Component | Amount (g) |
|---|---|
| Rapamycin | 20 |
| PEG-40 stearate | 150 |
| PEG-150 stearate | 50 |
| Miglyol 812 | 20 |

### Example 22

| Component | Amount (g) |
|---|---|
| Pioglitazone | 15 |
| Pureco 76 | 20 |
| Lutrol OP 2000 | 30 |
| PEG-100 hydrogenated castor oil | 100 |
| PEG-100 oleate (Crodet O-100) | 100 |
| Nonpareil seed (25/30 mesh) | 200 |

### Example 23

| Component | Amount (g) |
|---|---|
| Oxaprozin | 50 |
| Safflower oil | 25 |
| PEG-10 soya sterol (Nikkol BYS-20) | 25 |
| Myrj 52 | 150 |
| Nonpareil seed (25/30 mesh) | 300 |

### Example 24

| Component | Amount (g) |
|---|---|
| Tretinoin | 50 |
| Capmul GMO-K | 50 |
| Sodium taurocholate | 100 |
| DPPC | 50 |
| DMPC | 50 |

### Example 25

| Component | Amount (g) |
|---|---|
| Celecoxib | 50 |
| Myrj 52 | 100 |
| Glycerol monolaurate | 30 |
| Hydrogenated coconut oil | 20 |
| Nonpareil seed (25/30 mesh) | 200 |

### Example 26

| Component | Amount (g) |
|---|---|
| Rofecoxib | 10 |
| Kessco PEG 1540 MS (PEG-32 stearate) | 160 |
| Imwitor 312 | 20 |
| Hydrogenated palm oil (Softisan 154) | 20 |

### Example 27

| Component | Amount (g) |
|---|---|
| Fenofibrate | 100 |
| Imwitor 742 | 40 |
| Imwitor 988 | 40 |
| Sodium alginate | 4 |
| Crodet O-40 | 120 |
| Myrj 51 | 120 |
| Water | 20 |

### Example 28

| Component | Amount (g) |
|---|---|
| Saquinavir | 200 |
| HPMC | 50 |
| Myrj 52 | 130 |
| Arlacel 186 | 20 |

Pellets of various lansoprazole formulations were formed by spray congealing in example 29-34. The mean diameters of the pellets were 0.5-2 mm in average, preferably 0.8-1.2 mm. The compositions for making the active pellets are set forth below.

### Example 29

| Component | Amount (wt/wt) |
|---|---|
| Lansoprazole | 1.0 |
| Cremophor EL | 2.0 |
| PEG 4600 | 8.0 |

### Example 30

| Component | Amount (wt/wt) |
|---|---|
| Lansoprazole | 2.0 |
| Cremophor RH40 | 3.0 |
| PEG 6000 | 7.0 |
| Glycerol monostearate | 1.0 |

### Example 31

| Component | Amount (wt/wt) |
|---|---|
| Lansoprazole | 1.0 |
| Poloxamer 188 | 3.0 |
| PVP-K12 | 5.0 |
| Glyceryl behenate | 2.0 |
| Magnesium carbonate | 0.1 |

### Example 32

| Component | Amount (wt/wt) |
|---|---|
| Lansoprazole | 1.0 |
| TPGS | 6.0 |
| PEG 4600 | 4.0 |
| Calcium carbonate | 0.1 |

### Example 33

| Component | Amount (wt/wt) |
|---|---|
| Lansoprazole | 1.0 |
| Cremophor EL | 2.0 |
| PEG 4600 | 8.0 |
| Magnesium carbonate | 1.0 |

### Example 34

| Component | Amount (wt/wt) |
|---|---|
| Lansoprazole | 0.5 |
| Cremophor EL | 4.0 |
| PEG 6000 | 4.0 |
| Magnesium carbonate | 1.0 |

The pellets obtained from Examples 29, 32, 33 and 34 were seal coated with PVP-K30 (2-3% by weight of the pellet), hydrogenated vegetable oil (2-3% by weight of the pellet), glycerol monostearate (2-3% by weight of the pellet) or a first coat of glycerol monolaurate (1-2% by weight of the pellet) and magnesium carbonate (1-2% by weight of the pellet) and a second coat of glyceryl behenate (2-3% by weight of the pellet) in a coating pan or a fluidize bed.

The pellets of Examples 29-34 can be further enteric coated with the following enteric coating formulation:

| Component | Amount (wt/wt) |
|---|---|
| Hydroxypropylmethylcellulose phthalate | 20 |
| Cetyl alcohol | 1 |
| Talc | 10 |

The enteric coated materials are dissolved in an isopropyl alcohol/acetone mixture and spray coated onto the pellets in a coating pan or a fluidize bed.

Pellets of various active ingredient-containing formulations can be formed by spray congealing or spray coating onto an inert core, as shown in Examples 35, 36 and 37. The mean diameters of the pellets are 0.5-3 mm in average, preferably 0.8-2 mm. The compositions for making the active pellets are set forth below.

### Example 35

Amount (wt/wt)

Component A

| | |
|---|---|
| Zaleplon | 1.0 |
| PEG 4600 | 9.0 |

Component B

| | |
|---|---|
| Zaleplon | 1.0 |
| Gelucire 50/13 | 5.0 |
| Glyceryl monolaurate | 4.0 |

Pellets with different release rate of zaleplon from component A (immediate release) and component B (sustained release) are seal coated with PVP-K-30 (2-3% by of the pellet) containing different colorants to distinguish one from another. Pellets of component A and component B are mixed in a 1:2 weight ratio and filled in two piece capsules and each capsule contains 10 mg of zaleplon.

### Example 36

Amount (wt/wt)

Component A

| | |
|---|---|
| Sumatriptan | 1.5 |
| Gelucire 50/13 | 8.5 |

Component B

| | |
|---|---|
| Sumatriptan | 1.5 |
| Glyceryl monostearate | 8.5 |

Pellets with different release rates of sumatriptan from component A (immediate release) and component B (sustained release) are seal coated with PVP-K-30 (2-3% by of the pellet) containing different colorants to distinguish one from another. Pellets of component A and component B are mixed in a 1:3 weight ratio and filled in two piece capsules and each capsule contains 50 mg of sumatriptan.

### Example 37

| Component | Amount (wt/wt) |
|---|---|
| Sumatriptan | 1.0 |
| TPGS | 4.0 |
| PEG 6000 | 5.0 |

Pellets are seal coated either with PVP-K30 (2-3% by weight of the pellet) or with Eudragit S (4-5% by weight of the pellet). Pellets coated with PVP-K30 and pellets coated with Eudragit S are mixed in a 1:1 weight ratio and filled in two piece capsules and each capsule contains 40 mg of sumatriptan.

All patents, publications, and other published documents mentioned or referred to in this specification are herein incorporated by reference in their entirety.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A pharmaceutical composition in the form of a solid carrier comprising an admixture of:
a) a therapeutically effective amount of lansoprazole; and
b) at least one excipient selected from the group consisting of:
i) a hydrophilic surfactant;
ii) at least one lipophilic additive selected from the group consisting of lipophilic surfactants, triglycerides, and combinations thereof; and
iii) a solubilizer.

2. The pharmaceutical composition of claim 1 wherein the lansoprazole is processed by a treatment with an interfacial modifying agent selected from the group consisting of surfactants, polymers, lipids, gelatins, saccharides, and combinations thereof.

3. The pharmaceutical composition of claim 2 wherein the treatment comprises coating the lansoprazole with the interfacial modifying agent.

4. The pharmaceutical composition of claim 1 wherein the admixture comprises a hydrophilic surfactant.

5. The pharmaceutical composition of claim 4 wherein the hydrophilic surfactant is a non-ionic hydrophilic surfactant having an HLB value of at least about 10 and is selected from the group consisting of alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; tocopherol polyethylene glycol succinates; sugar esters; sugar ethers; sucroglycerides; and combinations thereof.

6. The pharmaceutical composition of claim 4 wherein the hydrophilic surfactant is an ionic surfactant selected from the group consisting of alkyl ammonium salts; bile acids and salts, analogues, and derivatives thereof; fatty acid derivatives of amino acids, carnitines, oligopeptides, and polypeptides; glyceride derivatives of amino acids, oligopeptides, and polypeptides; acyl lactylates; mono- and diacetylated tartaric acid esters of mono- and diglycerides; succinylated monoglycerides; citric acid esters of mono- and diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and combinations thereof.

7. The pharmaceutical composition of claim 1 wherein the admixture comprises a lipophilic additive.

8. The pharmaceutical composition of claim 7 wherein the lipophilic additive is a lipophilic surfactant selected from the group consisting of alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and combinations thereof.

9. The pharmaceutical composition of claim 7 wherein the lipophilic additive is a triglyceride selected from the group consisting of vegetable oils, fish oils, animal fats, hydrogenated vegetable oils, partially hydrogenated vegetable oils, synthetic triglycerides, modified triglycerides, fractionated triglycerides, and combinations thereof.

10. The pharmaceutical composition of claim 1 wherein the admixture comprises a solubilizer.

11. The pharmaceutical composition of claim 10 wherein the solubilizer is selected from the group consisting of alcohols and polyols; ethers of polyethylene glycols having an average molecular weight of about 200 to about 6000; amides; esters, and combinations thereof.

12. The pharmaceutical composition of claim 10 wherein the solubilizer is selected from the group consisting of polyvinylpyrrolidone, hydroxypropyl methylcellulose, hydroxypropyl cyclodextrins, polyethylene glycol, and combinations thereof.

13. The pharmaceutical composition of claim 1 wherein the admixture further comprises a bufferant selected from the group consisting of pharmaceutically acceptable bases, salts of pharmaceutically acceptable cations, and combinations thereof.

14. The pharmaceutical composition of claim 1 further comprising an anti-microbial agent.

15. The pharmaceutical composition of claim 14 wherein the anti-microbial agent is selected from the group consisting of amoxicillin, clarithromycin, erythromycin, metronidazole, tetracycline, and combinations thereof.

16. The pharmaceutical composition of claim 1 further comprising an antacid agent.

17. The pharmaceutical composition of claim 16 wherein the antacid agent is selected from the group consisting of aluminum hydroxide, magnesium hydroxide, sodium carbonate, calcium carbonate, and combinations thereof.

18. The pharmaceutical composition of claim 1 wherein the solid carrier further comprises a substrate and the admixture is coated on the substrate as an encapsulation coat.

19. The pharmaceutical composition of claim 1 wherein the solid carrier is a bead, beadlet, granule, spherule, pellet, microcapsule, microsphere, or nanosphere.

20. The pharmaceutical composition of claim 1 wherein the solid carrier is enteric coated.

21. The pharmaceutical composition of claim 1 wherein the solid carrier is seal coated with a material selected from the group consisting of lipophilic surfactants, triglycerides, waxes, polymers, and combinations thereof.

22. The pharmaceutical composition of claim 21 wherein the seal coat further comprises a bufferant selected from the group consisting of pharmaceutically acceptable bases, salts of pharmaceutically acceptable cations, and combinations thereof.

23. The pharmaceutical composition of claim 21 wherein the seal coat material is digestible.

24. The pharmaceutical composition of claim 1 which is in the form of a capsule, sachet, sprinkle, dry syrup, or strip.

25. The pharmaceutical composition of claim 24 wherein the capsule is a gelatin capsule, a hydroxypropylmethylcellulose capsule, or a starch capsule.

26. The pharmaceutical composition of claim 1 wherein the solid carrier is prepared by spray congealing process.

27. The pharmaceutical composition of claim 1 wherein the solid carrier is prepared by a process without the need of introducing water or organic solvents.

28. A method of administering lansoprazole or a pharmaceutically acceptable salt, isomer or derivative thereof, to an individual comprising orally administering to the individual a dosage form of the pharmaceutical composition of claim 1.

29. The method of claim 28 wherein the individual is being treated for at least one condition selected from the group consisting of duodenal ulcer, gastric ulcer, gastroesophageal reflux disease, erosive esophagitis, and pathological hypersecretory conditions.

30. A method of improving the oral bioavailability of lansoprazole, or a pharmaceutically acceptable salt, isomer or derivative thereof, in mammals under fed condition, comprising orally administering to the mammal a dosage form of the pharmaceutical composition of claim 1.

31. The method of claim 30 wherein the mammal is a human.

32. A method of improving the in vivo or ex vivo stability of an active ingredient at a pH within the range of about 1-6.8, wherein the active agent is selected from the group consisting of lansoprazole and pharmaceutically acceptable salts, isomers and derivatives thereof, in an acidic pH within thee range of about 1-6.8, comprising formulating the active ingredient in a pharmaceutical composition comprising at least one excipient selected from the group consisting of:
a) a hydrophilic surfactant;
b) at least one lipophilic additive selected from the group consisting of lipophilic surfactants, triglycerides, and combinations thereof, and
c) a solubilizer; and
optionally providing the pharmaceutical composition with a seal coat or an enteric coat.

33. A method of improving the stability of an active ingredient during storage, wherein in the active ingredient is selected from the group consisting of lansoprazole and pharmaceutically acceptable salts, isomers and derivatives thereof, comprising formulating the active ingredient in a pharmaceutical composition comprising at least one excipient selected from the group consisting of:
a) a hydrophilic surfactant;
b) at least one lipophilic additive selected from the group consisting of lipophilic surfactants, triglycerides, and combinations thereof; and
c) a solubilizer; and
optionally providing the pharmaceutical composition with a seal coat.

34. A pharmaceutical composition in the form of a solid carrier prepared by spray congealing comprising an admixture of:
a) a therapeutically effective amount of lansoprazole;
b) at least one hydrophilic surfactant;
c) a solubilizer;
wherein the solid carrier is seal coated with a material selected from the group consisting of lipophilic surfactants, triglycerides, waxes, polymers, and combinations thereof.

35. The pharmaceutical composition of claim 34 wherein the solid carrier is substantially free of bufferants selected from the group consisting of pharmaceutically acceptable bases, salts of pharmaceutically acceptable cations, and combinations thereof.

36. The pharmaceutical composition of claim 34 wherein the solid carrier is substantially free of hard fat.

37. A pharmaceutical composition in the form of a solid carrier comprising an admixture of:
a) a therapeutically effective amount of an active ingredient selected from the group consisting of esomeprazole, pantoprazole, rabeprazole, and pharmaceutically acceptable salts, isomers and derivatives thereof; and
b) at least one excipient selected from the group consisting of:
i) a hydrophilic surfactant;
ii) at least one lipophilic additive selected from the group consisting of lipophilic surfactants, triglycerides, and combinations thereof; and
iii) a solubilizer.

38. The pharmaceutical composition of claim 37 wherein the active ingredient is processed by a treatment with an interfacial modifying agent selected from the group consisting of surfactants, polymers, lipids, gelatins, saccharides, and combinations thereof.

39. The pharmaceutical composition of claim 38 wherein the treatment comprises coating the active ingredient with the interfacial modifying agent.

40. The pharmaceutical composition of claim 37 further comprising a bufferant selected from the group consisting of pharmaceutically acceptable bases, salts of pharmaceutically acceptable cations, and combinations thereof.

41. The pharmaceutical composition of claim 37 farther comprising an anti-microbial agent, an antacid agent, or combination thereof.

42. The pharmaceutical composition of claim 37 wherein the solid carrier further comprises a substrate, and the admixture is coated on the substrate as an encapsulation coat.

43. The pharmaceutical composition of claim 37 wherein the solid carrier is a bead, beadlet, granule, spherule, pellet, microcapsule, microsphere, or nanosphere.

44. The pharmaceutical composition of claim 37 wherein the solid carrier is enteric coated.

45. The pharmaceutical composition of claim 37 wherein the solid carrier is seal coated with a material selected from the group consisting of lipophilic surfactants, triglycerides, waxes, polymers, and combinations thereof.

46. The pharmaceutical composition of claim 45 wherein the seal coat further comprises a bufferant selected from the group consisting of pharmaceutically acceptable bases, salts of pharmaceutically acceptable cations, and combinations thereof.

47. The pharmaceutical composition of claim 37 is in the form of a capsule, tablet, effervescent tablet, sachet, sprinkle, dry syrup, or reconstitutable solid.

48. The pharmaceutical composition of claim 47 wherein the capsule is a gelatin capsule, a hydroxypropylmethylcellulose capsule or a starch capsule.

49. A method of administering an active ingredient selected from the group consisting of esomeprazole, pantoprazole, rabeprazole, and pharmaceutically acceptable salts, isomers and derivatives thereof, to an individual comprising orally administering to the individual a dosage form of the pharmaceutical composition of claim 37.

50. The method of claim 49 wherein the individual is being treated for at least one condition selected from the group consisting of duodenal ulcer, gastric ulcer, gastroesophageal reflux disease, erosive esophagitis, and pathological hypersecretory conditions.

51. A method of improving the oral bioavailability of an active ingredient selected from the group consisting of esomeprazole, pantoprazole, rabeprazole, and pharmaceutically acceptable salts, isomers and derivatives thereof, in mammals under fed condition, comprising orally administering to the mammal a dosage form of the pharmaceutical composition of claim 37.

52. The method of claim 51 wherein the mammal is a human.

53. A method of improving the in vivo or ex vivo stability of an active ingredient at a pH within the range of about 1-6.8, wherein the active agent is selected from the group consisting of esomeprazole, pantoprazole, rabeprazole, and pharmaceutically acceptable salts, isomers and derivatives thereof, comprising formulating the active ingredient in a pharmaceutical composition comprising at least one excipient selected from the group consisting of:
a) a hydrophilic surfactant;
b) at least one lipophilic additive selected from the group consisting of lipophilic surfactants, triglycerides, and combinations thereof; and
c) a solubilizer; and
optionally providing the pharmaceutical composition with a seal coat or an enteric coat.

54. A method of improving the stability of an active ingredient during storage, wherein the active ingredient selected from the group consisting of esomeprazole, pantoprazole, rabeprazole, and pharmaceutically acceptable salts, isomers and derivatives thereof, comprising formulating the active ingredient in pharmaceutical composition comprising at least one excipient selected from the group consisting of:
a) a hydrophilic surfactant;
b) at least one lipophilic additive selected from the group consisting of lipophilic surfactants, triglycerides, and combinations thereof; and
c) a solubilizer; and
optionally providing the pharmaceutical composition with a seal coat, wherein at least one of the lipophilic additive or seal coat reduces the permeation of moisture to the active ingredient.

55. The method of claim 54 wherein the pharmaceutical composition is further provided with an enteric coat.
